SEQUENCE LISTING (34 sequences)

SEQ ID NO: 1 - protein, 31 aa, Artificial Sequence
  Synthetic Construct
IKSLQNAINGLTNKINTLIEKINILFTACRR

SEQ ID NO: 2 - protein, 31 aa, Artificial Sequence
  Synthetic Construct
IENLNKKIDDLFLDIWTLNAEILVLLENCRR

SEQ ID NO: 3 - protein, 31 aa, Artificial Sequence
  Synthetic Construct
IRTLDFHISNLKNLIEKLKSQIKNLAKECRR

SEQ ID NO: 4 - protein, 31 aa, Artificial Sequence
  Synthetic Construct
ISGLNASIVNLQKEIDRLNEVIKNLNESCRR

SEQ ID NO: 5 - protein, 31 aa, Artificial Sequence
  Synthetic Construct
ILHLEGEINKLKSAILSLNKAIVSLSNGCRR

SEQ ID NO: 6 - protein, 31 aa, Artificial Sequence
  Synthetic Construct
ILSLNKAIVSLSNGISVLTSKILDLKNYCRR

SEQ ID NO: 7 - protein, 31 aa, Artificial Sequence
  Synthetic Construct
ISQLNEKINQLLAFIRKLDELIHNLNAGCRR

SEQ ID NO: 8 - protein, 31 aa, Artificial Sequence
  Synthetic Construct
IEKLKEAIRDLNKAIQSLQSSIGNLIVACRR

SEQ ID NO: 9 - protein, 31 aa, Artificial Sequence
  Synthetic Construct
IRDLNKAIQSLQSSIGNLIVAIKSLQDYCRR

SEQ ID NO: 10 - protein, 31 aa, Artificial Sequence
  Synthetic Construct
INKLKSDIEELKEWIRRLNQKIDSLGNWCRR

SEQ ID NO: 11 - protein, 31 aa, Artificial Sequence
  Synthetic Construct
INELAAAILNLKNAIQKLNAAIADLVQACRR

SEQ ID NO: 12 - protein, 31 aa, Artificial Sequence
  Synthetic Construct
IQKLNAAIADLVQAIQSLGTAIQALQDHCRR

SEQ ID NO: 13 - protein, 31 aa, Artificial Sequence
  Synthetic Construct
IAALNKSISDLLQHIAQLDTYISALTSACRR

SEQ ID NO: 14 - protein, 31 aa, Artificial Sequence
  Synthetic Construct
IQELAKLILTLKKAITELNEAIRDLANSCRR

SEQ ID NO: 15 - protein, 31 aa, Artificial Sequence
  Synthetic Construct
ITELNEAIRDLANSIKILVKMISALQNQCRR

SEQ ID NO: 16 - protein, 31 aa, Artificial Sequence
  Synthetic Construct
ILDLSTDINQLNQLIKSLEDHIQRLTDYCRR

SEQ ID NO: 17 - protein, 29 aa, Artificial Sequence
  Synthetic Construct
IXXLXXXIXXLXXXIXXLXXXIXXLXXXI

SEQ ID NO: 18 - protein, 29 aa, Human respiratory snynctial virus
VLHLEGEVNKIKSALLSTNKAVVSLSNGV

SEQ ID NO: 19 - protein, 29 aa, Artificial Sequence
  Synthetic Construct
ILHLEGEINKLKSAILSLNKAIVSLSNGI

SEQ ID NO: 20 - protein, 29 aa, Influenzavirus A
QKSTQNAINGITNKVNTVIEKMNIQFTAV

SEQ ID NO: 21 - protein, 29 aa, Influenzavirus A
MENLNKKVDDGFLDIWTYNAELLVLLENE

SEQ ID NO: 22 - protein, 29 aa, Influenzavirus A
ERTLDFHDSNVKNLYEKVKSQLKNNAKEI

SEQ ID NO: 23 - protein, 29 aa, Severe acute respiratory syndrome-related coronavirus
ISGINASVVNIQKEIDRLNEVAKNLNESL

SEQ ID NO: 24 - protein, 29 aa, Human respiratory syncytial virus
LLSTNKAVVSLSNGVSVLTSKVLDLKNYI

SEQ ID NO: 25 - protein, 29 aa, Human respiratory syncytial virus
ISQVNEKINQSLAFIRKSDELLHNVNAGK

SEQ ID NO: 26 - protein, 29 aa, Human parainfluenza virus 3
IEKLKEAIRDTNKAVQSVQSSIGNLIVAI

SEQ ID NO: 27 - protein, 29 aa, Human parainfluenza virus 3
IRDTNKAVQSVQSSIGNLIVAIKSVQDYV

SEQ ID NO: 28 - protein, 29 aa, Human parainfluenza virus 3
LNKAKSDLEESKEWIRRSNQKLDSIGNWH

SEQ ID NO: 29 - protein, 29 aa, Human parainfluenza virus 5
ANENAAAILNLKNAIQKTNAAVADVVQAT

SEQ ID NO: 30 - protein, 29 aa, Human parainfluenza virus 5
IQKTNAAVADVVQATQSLGTAVQAVQDHI

SEQ ID NO: 31 - protein, 29 aa, Human parainfluenza virus 5
LAAVNKSLSDALQHLAQSDTYLSAITSAT

SEQ ID NO: 32 - protein, 29 aa, Human parainfluenza virus 4
AQENAKLILTLKKAATETNEAVRDLANSN

SEQ ID NO: 33 - protein, 29 aa, Human parainfluenza virus 4
ATETNEAVRDLANSNKIVVKMISAIQNQI

SEQ ID NO: 34 - protein, 29 aa, Human parainfluenza virus 4
PLDLSTDLNQYNQLLKSAEDHIQRSTDYL

(19) United States
(12) Patent Application Publication
(10) Pub. No.: US 2012/0009212 A1
(43) Pub. Date: Jan. 12, 2012

(54) CONJUGATES UTILIZING PLATFORM TECHNOLOGY FOR STIMULATING IMMUNE RESPONSE

(75) Inventors: Robert S. HODGES, Denver, CO (US); Kathryn V. Holmes, Golden, CO (US); Zhe Yan, Lakewood, CO (US); Wendy J. Hartsock, Aurora, CO (US); Zhaohui Qian, Colorado Springs, CO (US); Brooke Elizabeth Bishop Hirsch, Broomfield, CO (US)

(73) Assignee: The Regents of the University of Colorado, a body corporate

(21) Appl. No.: 13/014,664

(22) Filed: Jan. 26, 2011

Related U.S. Application Data
(60) Provisional application No. 61/298,354, filed on Jan. 26, 2010.

(51) Int. Cl. A61K 39/385; A61P 31/12; A61P 37/04; C07K 17/02; C07K 17/10

(52) U.S. Cl. 424/196.11; 530/405; 530/363; 530/367; 530/395

(57) ABSTRACT

Templated conjugates created from naturally-occurring protein sequences found in pathogens, such as viruses, are disclosed. The sequences are “templated” into a consensus coiled-coil sequence in order to form a two-stranded antigen suitable for immunization of a subject.

### CROSS-REFERENCE TO RELATED APPLICATIONS

This application claims priority benefit under 35 U.S.C. §119(e) to U.S. Provisional Patent Application No. 61/298,354, filed Jan. 26, 2010. The content of that application is hereby incorporated by reference herein in its entirety.

### TECHNICAL FIELD OF THE INVENTION

The application relates to technology for producing conjugates effective for stimulating an immune response against a wide variety of viral diseases.

### BACKGROUND OF THE INVENTION

Infectious diseases such as influenza infect hundreds of millions of people annually. Worldwide, influenza can affect up to 5-15% of the population, with an estimated three million to five million cases of severe illness, and an estimated 250,000 to 500,000 deaths every year. (See URL www.who.int/mediacentre/factsheets/2003/fs211/en/). Other viral pathogens, such as severe acute respiratory syndrome (SARS) virus, parainfluenza virus, and respiratory syncytial virus, inflict additional morbidity and mortality annually.

Controlling these diseases is complicated by mutations in the pathogens, such as the constant antigenic drift and periodic antigenic shift of the influenza virus. Transmission of the diseases increases as the mutations cause accumulation of antigenic changes from previously circulating influenza virus strains, so that the mutant viruses encounter individuals who do not have protective antibodies against the mutant virus strain. The rapid selection of mutant influenza viruses by immune pressure requires development and production of new influenza vaccines every year.

The need for an effective vaccine strategy has been noted by several researchers. Nabel and Fauci comment on the need for a broadly protective “universal influenza vaccine” in Nature Medicine, 16(12):1389 (2010). Tripet et al. describe one approach to peptide vaccines in “Template-based coiled-coil antigens elicit neutralizing antibodies to the SARS-coronavirus,” Journal of Structural Biology, 155:176-194 (2006), and in International Patent Application WO 2005/077103. Wrammer et al. describe neutralizing antibodies that cross-reacted with multiple strains of influenza in “Broadly cross-reactive antibodies dominate the human B cell response against 2009 pandemic H1N1 influenza virus infection,” J. Experimental Medicine, 2011 Jan. 10, Epub ahead of print, PMID: 21220454.

The current invention addresses the need for effective vaccines against pathogens such as respiratory viruses. The invention also addresses the need to protect against a rapidly mutating pathogen, multiple antigenically distinct strains of a single pathogen, or multiple pathogens with a single vaccine.

### BRIEF SUMMARY OF THE INVENTION

The invention encompasses templated conjugates of two peptides. The conjugate is produced by adapting a first amino acid sequence of a naturally occurring alpha helical epitope into a heptad repeat to form a first templated epitope; adapting a second sequence of a naturally occurring alpha helical epitope into a heptad repeat to form a second templated epitope; forming a complex of the two templated epitopes to create a coiled-coil structure; and linking the coiled-coil structure to a carrier, such as a carrier protein, to form the conjugate. In one embodiment, the two templated epitopes have different sequences. The invention also encompasses a method of generating an immune response by administering the conjugate to a subject, such as a subject in need thereof. The conjugate is administered to the subject in a sufficient amount to create a protective immune response in the subject. In one embodiment, at least one of the epitopes is not derived from an influenza virus protein.

In one embodiment, the conjugate comprises two polypeptides, that is, a first polypeptide and a second polypeptide, wherein each polypeptide comprises at least one heptad repeat, and wherein the two polypeptides have less than, or no more than, about 90% sequence identity; a covalent linkage between the two polypeptides; and a carrier, such as a carrier protein, covalently linked to one of the polypeptides. In another embodiment, the conjugate comprises two polypeptides, that is, a first polypeptide and a second polypeptide, wherein each polypeptide comprises at least one heptad repeat, and wherein the two polypeptides have about 100% sequence identity; a covalent linkage between the two polypeptides; and a carrier, such as a carrier protein, covalently linked to one of the polypeptides. In any of the embodiments, the conjugate comprises at least two heptad repeats. In any of the embodiments, the conjugate comprises at least three heptad repeats. In any of the embodiments, the conjugate comprises at least four heptad repeats. In any of the embodiments, the conjugate comprises at least five heptad repeats. In any of the embodiments, the conjugate comprises at least six heptad repeats. In any of the embodiments, the conjugate comprises at least seven heptad repeats. In any of the embodiments, the conjugate comprises at least eight heptad repeats. In any of the embodiments, the conjugate comprises at least nine heptad repeats. In any of the embodiments, the conjugate comprises at least ten heptad repeats. In any of the embodiments, the conjugate comprises at least eleven heptad repeats. In any of the embodiments, the conjugate comprises at least twelve heptad repeats. In any of the embodiments, the conjugate comprises at least thirteen heptad repeats. In any of the embodiments, the conjugate comprises at least fourteen heptad repeats. In any of the embodiments, the conjugate comprises at least fifteen heptad repeats. In additional embodiments, a single additional isoleucine residue occurs immediately after the last heptad repeat. In any of the embodiments, at least one of the epitopes is not derived from an influenza virus protein.

In one embodiment, the first polypeptide of the conjugate can comprise the form:

[I-b₂ᵢ-c₂ᵢ-L-e₂ᵢ-g₂ᵢ]ₙ,

where [I-b₂ᵢ-c₂ᵢ-L-e₂ᵢ-g₂ᵢ] is a segment that repeats n times in the sequence of the first polypeptide. “I” in each segment is isoleucine and “L” in each segment is leucine. The number n is an integer of at least 3. In some embodiments, n is an integer of from 3 to 15, inclusive; that is, n is 3, 4, 5, 6, 7, 8, 9, 10, 11, 12, 13, 14, or 15.

The number i is an integer from 1 to n, wherein the value of i is determined by the position of the segment in which it appears. The N-terminal segment which appears first in the sequence is assigned a value of i=1. The number i is incremented by one for each additional segment, until the C-terminal segment is assigned a value of i=n. Each b, c, e, f, and g in each of the n segments can be selected independently of each b, c, e, f, and g amino acid in all other segments of the first polypeptide, and of all segments of the second polypeptide. In one embodiment, the b, c, e, f, and g amino acids are selected from an alpha helical region of a Class 1 viral fusion protein of a pathogen against which an immune response is desired. In additional embodiments, a single additional isoleucine residue occurs immediately after the last segment (i.e., at the C-terminus of the last segment).

In one embodiment, the second polypeptide of the conjugate can comprise the form:

[I-b₂ᵢ-c₂ᵢ-L-e₂ᵢ-g₂ᵢ]ₙ,

where [I-b₂ᵢ-c₂ᵢ-L-e₂ᵢ-g₂ᵢ] is a segment that repeats n times in the sequence of the second polypeptide. “I” in each segment is isoleucine and “L” in each segment is leucine. The number n is an integer of at least 3 and is the same as n for the first polypeptide. In some embodiments, n is an integer of from 3 to 15, inclusive; that is, n is 3, 4, 5, 6, 7, 8, 9, 10, 11, 12, 13, 14, or 15, and is the same as n for the first polypeptide. The number i is an integer from 1 to n, wherein the value of i is determined by the position of the segment in which it appears, such that the N-terminal segment which appears first in the sequence is assigned a value of i=1, i is incremented by one for each additional segment, and the C-terminal segment is assigned a value of i=n. Each b, c, e, f, and g in each of the n segments is selected independently of each b, c, e, f, and g amino acid in all other segments of the second polypeptide, and of all segments of the first polypeptide. The b, c, e, f, and g amino acids are selected from an alpha helical region of a Class 1 viral fusion protein of a pathogen against which an immune response is desired. In one embodiment, the second polypeptide of the conjugate has the same sequence as the first polypeptide of the conjugate. In another embodiment, the second polypeptide of the conjugate has a different sequence from the first polypeptide of the conjugate, and the b, c, e, f, and g amino acids are either selected from a different Class 1 viral fusion protein than the protein from which the first polypeptide is selected, or are selected from a different portion of the same Class 1 viral fusion protein that the region from which the first polypeptide is selected. In additional embodiments, a single additional isoleucine residue occurs immediately after the last segment (i.e., at the C-terminus of the last segment). In one embodiment, the first polypeptide and the second polypeptide are of equal length.

In another embodiment, the invention embraces a conjugate of the form:
[Carrier Moiety]-[Linker A]-[Linker B]-[Templated Epitope 1]-[Epitope 1 Modifier][Templated Epitope 2]-[Epitope 2 Modifier]
   where Linker A, Linker B, Epitope 1 Modifier, and Epitope 2 Modifier are optionally present. In further embodiments, the conjugate can optionally comprise an additional covalent Linker C between Templated Epitope 1 and Templated Epitope 2; optionally comprise an additional covalent Linker D between Epitope 1 Modifier and Epitope 2 Modifier, or optionally comprise both an additional covalent Linker C between Templated Epitope 1 and Templated Epitope 2 and an additional covalent Linker D between Epitope 1 Modifier and Epitope 2 Modifier.

In one embodiment of the conjugate, the Epitope 1 Modifier and the Epitope 2 Modifier are both present and are selected from hydrophilic, polar, and charged amino acids. The Epitope 1 Modifier and the Epitope 2 Modifier can comprise one cysteine residue each, for use in forming a disulfide bond between Templated Epitope 1 and Templated Epitope 2 (such a disulfide bond would then comprise Linker D between the Epitope 1 Modifier and the Epitope 2 Modifier). The Epitope 1 Modifier and the Epitope 2 Modifier can be the same or different, and can be chosen from -Arg, -(Arg)₂, -(Arg)₃, -(Arg)₄, -Lys, -(Lys)₂, -(Lys)₃, -(Lys)₄, -Arg-amide, -(Arg)₂-amide, -(Arg)₃-amide, -(Arg)₄-amide, -Lys-amide, -(Lys)₂-amide, -(Lys)₃-amide, -(Lys)₄-amide, —Cys-Arg, -Cys-(Arg)₂, -Cys-(Arg)₃, -Cys-(Arg)₄, -Cys-Lys, -Cys-(Lys)₂, -Cys-(Lys)₃, -Cys-(Lys)₄, -Cys-Arg-amide, -Cys-(Arg)₂-amide, —Cys-(Arg)₃-amide, —Cys-(Arg)₄-amide, —Cys-Lys-amide, —Cys-(Lys)₂-amide, —Cys-(Lys)₃-amide, and —Cys-(Lys)₄-amide.

When [Linker A] is present, it can be a peptide; a non-genetically-coded amino acid such as norleucine, alpha-amino-3-guanidino propionic acid, or beta-alanine; or a peptide comprising a non-genetically-coded amino acid. When [Linker B] is present, it can be an amino acid or a peptide, such as -Gly-, -Gly-Gly-, -(Gly)₃-, and -(Gly)₄-.

In one embodiment, the epitopes used in the conjugates or modified or templated for use in the conjugates are derived from the stem region of a Class 1 viral fusion protein from one or more viruses having a Class 1 viral fusion protein. In one embodiment, the one or more viruses are selected from the group comprising influenza A virus strains, SARS virus, Respiratory Syncytial Virus, Parainfluenza Virus 5, Parainfluenza Virus 4, or Parainfluenza Virus 3. In one embodiment, the one or more viruses are selected from the group comprising SARS virus, Respiratory Syncytial Virus, Parainfluenza Virus 5, Parainfluenza Virus 4, or Parainfluenza Virus 3. In one embodiment, the Class 1 viral fusion protein can be selected from the group consisting of Influenza PR8 (Influenza A/PR/8/34 (H₁N₁)) HA₂ domain, SARS Coronavirus S2, Respiratory Syncytial Virus RSV A2 F, Parainfluenza Virus 3 PIV 3 F, Parainfluenza Virus 5 PIV 5 F, or Parainfluenza Virus 4 PIV 4A F. In another embodiment, the Class 1 viral fusion protein can be selected from the group consisting of SARS Coronavirus S2, Respiratory Syncytial Virus RSV A2 F, Parainfluenza Virus 3 PIV 3 F, Parainfluenza Virus 5 PIV 5 F, or Parainfluenza Virus 4 PIV 4A F. A heptad repeat region of the viral protein is selected as the epitope to be modified and templated for use in the conjugate.

The templated epitopes for use in the invention as Templated Epitope 1 and Templated Epitope 2 can be selected from the group consisting of:

Influenza PR8HA₂ 3 MP(381-409) Templated Epitope 3 MP (IKSLQNAINGLTNKINTLIEKINILFTACRR-amide (SEQ ID NO:1));
Influenza PR8HA₂ 5P(420-448) Templated Epitope 5P (IENLNKKIDDLFLDIWTLNAEILVLLENCRR-amide (SEQ ID NO: 2));
Influenza PR8HA₂ 6P(448-476) Templated Epitope 6P (IRTLDFHISNLKNLIEKLKSQIKNLAKECRR-amide (SEQ ID NO:3));
SARS Coronavirus HRC domain in S2 (1151-1179) Templated Epitope (ISGLNASIVNLQKEIDRLNEVIKNLNESCRR-amide (SEQ ID NO: 4));
Respiratory Syncytial Virus RSV A2 F(157-185) Templated Epitope (ILHLEGEINKLKSAILSLNKAIVSLSNGCRR-amide (Seq Id No: 5)),
Respiratory Syncytial Virus RSV A2 F(171-199) Templated Epitope (ILSLNKAIVSLSNGISVLTSKILDLKNYCRR-amide (SEQ ID NO:6));
Respiratory Syncytial Virus RSV A2 F(492-520) Templated Epitope (ISQLNEKINQLLAFIRKLDELIHNLNAGCRR-amide (SEQ ID NO:7));
Parainfluenza Virus 3 PIV 3 F(144-172) Templated Epitope (IEKLKEAIRDLNKAIQSLQSSIGNLIVACRR-amide (SEQ ID NO:8));
Parainfluenza Virus 3 PIV 3 F(151-179) Templated Epitope (IRDLNKAIQSLQSSIGNLIVAIKSLQDYCRR-amide (SEQ ID NO: 9));
Parainfluenza Virus 3 PIV 3 F(460-488) Templated Epitope (INKLKSDIEELKEWIRRLNQKIDSLGNWCRR-amide (SEQ ID NO: 10));
Parainfluenza Virus 5 PIV 5 F(130-158) Templated Epitope (INELAAAILNLKNAIQKLNAAIADLVQACRR-amide (SEQ ID NO: 11));
Parainfluenza Virus 5 PIV 5 F(144-172) Templated Epitope (IQKLNAAIADLVQAIQSLGTAIQALQDHCRR-amide (SEQ ID NO:12));
Parainfluenza Virus 5 PIV 5 F(453-481) Templated Epitope (IAALNKSISDLLQHIAQLDTYISALTSACRR-amide (SEQ ID NO:13));
Parainfluenza Virus 4 PIV 4A F(131-159) Templated Epitope (IQELAKLILTLKKAITELNEAIRDLANSCRR-amide (SEQ ID NO:14));
Parainfluenza Virus 4 PIV 4A F(145-173) Templated Epitope (ITELNEAIRDLANSIKILVKMISALQNQCRR-amide (SEQ ID NO:15)); and
Parainfluenza Virus 4 PIV 4A F(447-475) Templated Epitope (ILDLSTDINQLNQLIKSLEDHIQRLTDYCRR-amide (SEQ ID NO: 16)). In one embodiment, Templated Epitope 1 and Templated Epitope 2 are not identical (when non-identical Templated Epitope 1 and Templated Epitope 2 are used in a conjugate, the conjugate is then a hetero two-stranded conjugate). In another embodiment, only one of Templated Epitope 1 or Templated Epitope 2 is selected from an influenza virus epitope, and the other Templated Epitope is selected from a different virus. In another embodiment, Templated Epitope 1 and Templated Epitope 2 are identical (when identical Templated Epitope 1 and Templated Epitope 2 are used in a conjugate, the conjugate is then a homo two-stranded conjugate).

The templated epitopes for use in the invention as Templated Epitope 1 and Templated Epitope 2 can be selected from the group consisting of:

SARS Coronavirus HRC domain of S2 (1151-1179) Templated Epitope (ISGLNASIVNLQKEIDRLNEVIKNLNESCRR-amide (SEQ ID NO:4));
Respiratory Syncytial Virus RSV A2 F(157-185) Templated Epitope (ILHLEGEINKLKSAILSLNKAIVSLSNGCRR-amide (SEQ ID NO: 5)),
Respiratory Syncytial Virus RSV A2 F(171-199) Templated Epitope (ILSLNKAIVSLSNGISVLTSKILDLKNYCRR-amide (SEQ ID NO: 6));
Respiratory Syncytial Virus RSV A2 F(492-520) Templated Epitope (ISQLNEKINQLLAFIRKLDELIHNLNAGCRR-amide (SEQ ID NO:7));
Parainfluenza Virus 3 PIV 3 F(144-172) Templated Epitope (IEKLKEAIRDLNKAIQSLQSSIGNLIVACRR-amide (SEQ ID NO: 8));
Parainfluenza Virus 3 PIV 3 F(151-179) Templated Epitope (IRDLNKAIQSLQSSIGNLIVAIKSLQDYCRR-amide (SEQ ID NO: 9));
Parainfluenza Virus 3 PIV 3 F(460-488) Templated Epitope (INKLKSDIEELKEWIRRLNQKIDSLGNWCRR-amide (SEQ ID NO:10));
Parainfluenza Virus 5 PIV 5 F(130-158) Templated Epitope (INELAAAILNLKNAIQKLNAAIADLVQACRR-amide (SEQ ID NO:11));
Parainfluenza Virus 5 PIV 5 F(144-172) Templated Epitope (IQKLNAAIADLVQAIQSLGTAIQALQDHCRR-amide (SEQ ID NO: 12));
Parainfluenza Virus 5 PIV 5 F(453-481) Templated Epitope (IAALNKSISDLLQHIAQLDTYISALTSACRR-amide (SEQ ID NO:13));
Parainfluenza Virus 4 PIV 4A F(131-159) Templated Epitope (IQELAKLILTLKKAITELNEAIRDLANSCRR-amide (SEQ ID NO:14));
Parainfluenza Virus 4 PIV 4A F(145-173) Templated Epitope (ITELNEAIRDLANSIKILVKMISALQNQCRR-amide (SEQ ID NO:15)); and
Parainfluenza Virus 4 PIV 4A F(447-475) Templated Epitope (ILDLSTDINQLNQLIKSLEDHIQRLTDYCRR-amide (SEQ ID NO: 16)). In one embodiment, Templated Epitope 1 and Templated Epitope 2 are not identical. In another embodiment, Templated Epitope 1 and Templated Epitope 2 are identical.

In another embodiment, either Templated Epitope 1, Templated Epitope 2, or both Templated Epitope 1 and Templated Epitope 2 can be selected from the group consisting of:

### Influenza PR8HA₂ 3 MP(381-409) Templated Epitope 3 MP,

### Influenza PR8HA₂ 5P(420-448) Templated Epitope 5P;

### Influenza PR8HA₂ 6P(448-476) Templated Epitope 6P,

SARS Coronavirus HRC domain of S2 (1151-1179) Templated Epitope;

### Respiratory Syncytial Virus RSV A2 F(157-185) Templated Epitope,

### Respiratory Syncytial Virus RSV A2 F(171-199) Templated Epitope;

### Respiratory Syncytial Virus RSV A2 F(492-520) Templated Epitope;

### Parainfluenza Virus 3 PIV 3 F(144-172) Templated Epitope;

### Parainfluenza Virus 3 PIV 3 F(151-179) Templated Epitope;

### Parainfluenza Virus 3 PIV 3 F(460-488) Templated Epitope;

### Parainfluenza Virus 5 PIV 5 F(130-158) Templated Epitope;

### Parainfluenza Virus 5 PIV 5 F(144-172) Templated Epitope;

### Parainfluenza Virus 5 PIV 5 F(453-481) Templated Epitope;

### Parainfluenza Virus 4 PIV 4A F(131-159) Templated Epitope;

### Parainfluenza Virus 4 PIV 4A F(145-173) Templated Epitope; and

### Parainfluenza Virus 4 PIV 4A F(447-475) Templated Epitope

where the selected Templated Epitope has a free carboxy terminus (i.e., the sequence lacks a C-terminal amide). In one embodiment, Templated Epitope 1 and Templated Epitope 2 are not identical. In another embodiment, only one of Templated Epitope 1 or Templated Epitope 2 is selected from an influenza virus epitope, and the other Templated Epitope is selected from a different virus. In another embodiment, Templated Epitope 1 and Templated Epitope 2 are identical.

In another embodiment, either Templated Epitope 1, Templated Epitope 2, or both Templated Epitope 1 and Templated Epitope 2 can be selected from the group consisting of:

SARS Coronavirus HRC domain of S2 (1151-1179) Templated Epitope;

### Respiratory Syncytial Virus RSV A2 F(157-185) Templated Epitope,

### Respiratory Syncytial Virus RSV A2 F(171-199) Templated Epitope;

### Respiratory Syncytial Virus RSV A2 F(492-520) Templated Epitope;

### Parainfluenza Virus 3 PIV 3 F(144-172) Templated Epitope;

### Parainfluenza Virus 3 PIV 3 F(151-179) Templated Epitope;

### Parainfluenza Virus 3 PIV 3 F(460-488) Templated Epitope;

### Parainfluenza Virus 5 PIV 5 F(130-158) Templated Epitope;

### Parainfluenza Virus 5 PIV 5 F(144-172) Templated Epitope;

### Parainfluenza Virus 5 PIV 5 F(453-481) Templated Epitope;

### Parainfluenza Virus 4 PIV 4A F(131-159) Templated Epitope;

### Parainfluenza Virus 4 PIV 4A F(145-173) Templated Epitope; and

### Parainfluenza Virus 4 PIV 4A F(447-475) Templated Epitope

where the selected Templated Epitope has a free carboxy terminus (i.e., the sequence lacks a C-terminal amide). In one embodiment, Templated Epitope 1 and Templated Epitope 2 are not identical.

In another embodiment of the conjugate,

### Templated Epitope 1 is Influenza PR8HA₂ 3 MP(381-409) Templated Epitope 3 MP and Templated Epitope 2 is Influenza PR8HA₂ 5P(420-448) Templated Epitope 5P.

In another embodiment of the conjugate,

### Templated Epitope 1 is Influenza PR8HA₂ 3 MP(381-409) Templated Epitope 3 MP and Templated Epitope 2 is Influenza PR8HA₂ 6P(448-476) Templated Epitope 6P.

In another embodiment of the conjugate,

### Templated Epitope 1 is Influenza PR8HA₂ 5P(420-448) Templated Epitope 5P and Templated Epitope 2 is Influenza PR8HA₂ 6P(448-476) Templated Epitope 6P.

In another embodiment of the conjugate,

Templated Epitope 1 is Influenza PR8HA₂ 3 MP(381-409) Templated Epitope 3 MP and Templated Epitope 2 is SARS Coronavirus HRC domain of S2 (1151-1179) Templated Epitope.

In another embodiment of the conjugate,

Templated Epitope 1 is Influenza PR8HA₂ 5P(420-448) Templated Epitope 5P and Templated Epitope 2 is SARS Coronavirus HRC domain of S2 (1151-1179) Templated Epitope.

In another embodiment of the conjugate,

Templated Epitope 1 is Influenza PR8HA₂ 6P(448-476) Templated Epitope 6P and Templated Epitope 2 is SARS Coronavirus HRC domain of S2 (1151-1179) Templated Epitope.

In another embodiment of the conjugate,

### Templated Epitope 1 is Respiratory Syncytial Virus RSV A2 F(157-185) Templated Epitope and Templated Epitope 2 is Respiratory Syncytial Virus RSV A2 F(171-199) Templated Epitope.

In another embodiment of the conjugate,

### Templated Epitope 1 is Respiratory Syncytial Virus RSV A2 F(157-185) Templated Epitope and Templated Epitope 2 is Respiratory Syncytial Virus RSV A2 F(492-520) Templated Epitope.

In another embodiment of the conjugate,

### Templated Epitope 1 is Respiratory Syncytial Virus RSV A2 F(171-199) Templated Epitope and Templated Epitope 2 is Respiratory Syncytial Virus RSV A2 F(492-520) Templated Epitope.

In another embodiment of the conjugate,

### Templated Epitope 1 is Parainfluenza Virus 3 PIV 3 F(144-172) Templated Epitope and Templated Epitope 2 is Parainfluenza Virus 3 PIV 3 F(151-179) Templated Epitope.

In another embodiment of the conjugate,

### Templated Epitope 1 is Parainfluenza Virus 3 PIV 3 F(144-172) Templated Epitope and Templated Epitope 2 is Parainfluenza Virus 3 PIV 3 F(460-488) Templated Epitope.

In another embodiment of the conjugate,

### Templated Epitope 1 is Parainfluenza Virus 3 PIV 3 F(151-179) Templated Epitope and Templated Epitope 2 is Parainfluenza Virus 3 PIV 3 F(460-488) Templated Epitope.

In another embodiment of the conjugate,

### Templated Epitope 1 is Parainfluenza Virus 3 PIV 3 F(144-172) Templated Epitope and Templated Epitope 2 is Respiratory Syncytial Virus RSV A2 F(157-185) Templated Epitope.

In another embodiment of the conjugate,

### Templated Epitope 1 is Parainfluenza Virus 3 PIV 3 F(144-172) Templated Epitope and Templated Epitope 2 is Respiratory Syncytial Virus RSV A2 F(171-199) Templated Epitope.

In another embodiment of the conjugate,

### Templated Epitope 1 is Parainfluenza Virus 3 PIV 3 F(151-179) Templated Epitope and Templated Epitope 2 is Respiratory Syncytial Virus RSV A2 F(171-199) Templated Epitope.

In another embodiment of the conjugate,

### Templated Epitope 1 is Parainfluenza Virus 3 PIV 3 F(151-179) Templated Epitope and Templated Epitope 2 is Respiratory Syncytial Virus RSV A2 F(157-185) Templated Epitope.

In another embodiment of the conjugate,

### Templated Epitope 1 is Parainfluenza Virus 5 PIV 5 F(130-158) Templated Epitope and Templated Epitope 2 is Parainfluenza Virus 5 PIV 5 F(144-172) Templated Epitope.

In another embodiment of the conjugate,

### Templated Epitope 1 is Parainfluenza Virus 5 PIV 5 F(130-158) Templated Epitope and Templated Epitope 2 is Parainfluenza Virus 5 PIV 5 F(453-481) Templated Epitope.

In another embodiment of the conjugate,

### Templated Epitope 1 is Parainfluenza Virus 5 PIV 5 F(144-172) Templated Epitope and Templated Epitope 2 is Parainfluenza Virus 5 PIV 5 F(453-481) Templated Epitope.

In another embodiment of the conjugate,

### Templated Epitope 1 is Parainfluenza Virus 5 PIV 5 F(130-158) Templated Epitope and Templated Epitope 2 is Respiratory Syncytial Virus RSV A2 F(157-185) Templated Epitope.

In another embodiment of the conjugate,

### Templated Epitope 1 is Parainfluenza Virus 5 PIV 5 F(130-158) Templated Epitope and Templated Epitope 2 is Respiratory Syncytial Virus RSV A2 F(171-199) Templated Epitope.

In another embodiment of the conjugate,

### Templated Epitope 1 is Parainfluenza Virus 5 PIV 5 F(144-172) Templated Epitope and Templated Epitope 2 is Respiratory Syncytial Virus RSV A2 F(171-199) Templated Epitope.

In another embodiment of the conjugate,

### Templated Epitope 1 is Parainfluenza Virus 5 PIV 5 F(144-172) Templated Epitope and Templated Epitope 2 is Respiratory Syncytial Virus RSV A2 F(157-185) Templated Epitope.

In another embodiment of the conjugate,

### Templated Epitope 1 is Parainfluenza Virus 4 PIV 4A F(131-159) Templated Epitope and Templated Epitope 2 is Parainfluenza Virus 4 PIV 4A F(145-173) Templated Epitope.

In another embodiment of the conjugate,

### Templated Epitope 1 is Parainfluenza Virus 4 PIV 4A F(131-159) Templated Epitope and Templated Epitope 2 is Parainfluenza Virus 4 PIV 4A F(447-475) Templated Epitope.

In another embodiment of the conjugate,

### Templated Epitope 1 is Parainfluenza Virus 4 PIV 4A F(145-173) Templated Epitope and Templated Epitope 2 is Parainfluenza Virus 4 PIV 4A F(447-475) Templated Epitope.

In another embodiment of the conjugate,

### Templated Epitope 1 is Parainfluenza Virus 4 PIV 4A F(131-159) Templated Epitope and Templated Epitope 2 is Respiratory Syncytial Virus RSV A2 F(157-185) Templated Epitope.

In another embodiment of the conjugate,

### Templated Epitope 1 is Parainfluenza Virus 4 PIV 4A F(131-159) Templated Epitope and Templated Epitope 2 is Respiratory Syncytial Virus RSV A2 F(171-199) Templated Epitope.

In another embodiment of the conjugate,

### Templated Epitope 1 is Parainfluenza Virus 4 PIV 4A F(145-173) Templated Epitope and Templated Epitope 2 is Respiratory Syncytial Virus RSV A2 F(171-199) Templated Epitope.

In another embodiment of the conjugate,

### Templated Epitope 1 is Parainfluenza Virus 4 PIV 4A F(145-173) Templated Epitope and Templated Epitope 2 is Respiratory Syncytial Virus RSV A2 F(157-185) Templated Epitope.

In another embodiment of the invention, the carrier moiety of the conjugate is a protein. The protein can be keyhole limpet hemocyanin (KLH), bovine serum albumin (BSA), ovalbumin, tetanus toxoid, cholera subunit B, protein D from H. influenza, or diphtheria toxoid. In another embodiment of the invention, the carrier moiety of the conjugate is a non-proteinaceous moiety. The non-proteinaceous moiety can be a polysaccharide, such as alginic acid (alginate).

In another embodiment of the invention, the linkage between the carrier moiety and Linker A (if present), Linker B (if Linker B is present and Linker A is absent), or Templated Epitope 1 (if Linker A and Linker B are absent) is chemically definite.

In one embodiment of the conjugates, the templated epitopes used exclude templated influenza epitopes where both Templated Epitope 1 and Templated Epitope 2 have the same sequence.

In one embodiment of the conjugates, the templated epitopes used exclude templated influenza epitopes.

In any of the embodiments of the peptides, epitopes, and Templated Epitopes described herein, one, two, or three of the residues at the “a” or “d” position may be changed from the residues indicated. In one embodiment, one “a” residue is selected from an amino acid other than isoleucine. In one embodiment, two “a” residues are independently selected from amino acids other than isoleucine. In one embodiment, three “a” residues are independently selected from amino acids other than isoleucine. In one embodiment, one “d” residue is selected from an amino acid other than leucine. In one embodiment, two “d” residues are independently selected from amino acids other than leucine. In one embodiment, three “d” residues are independently selected from amino acids other than leucine. In one embodiment, one or two “a” residues are independently selected from an amino acid other than isoleucine and one “d” residue is independently selected from an amino acid other than leucine. In one embodiment, one “a” residue is independently selected from an amino acid other than isoleucine and one or two “d” residues are independently selected from an amino acid other than leucine.

In one embodiment, the invention embraces kits comprising a composition comprising a conjugate of the invention and instructions for use in a subject.

In one embodiment, the invention embraces a method of inducing an antibody response in an individual in need thereof, the method comprising administering any of the conjugates as disclosed herein to an individual in need thereof, in an amount sufficient to induce an antibody response in the individual. In one embodiment, the antibody response is the production of a neutralizing antibody.

### BRIEF DESCRIPTION OF THE DRAWINGS

FIG. 1 is a schematic diagram of the templated conjugate. (A) with optional Linker C; (B) with optional Linker D.

FIG. 2 shows the arrangement of residues in the coiled-coil structure.

FIG. 3 depicts native sequences (A) used to create 3 hetero two stranded templated peptide conjugates (B) derived from influenza virus PR8

FIG. 4 depicts native sequences (A) used to create a homo two stranded templated peptide conjugate (B) derived from Severe Acute Respiratory Syndrome (SARS) coronavirus.

FIG. 5 depicts native sequences (A) used to create 3 hetero two stranded templated peptide conjugates (B) derived from a combination of influenza and SARS virus.

FIG. 6 depicts native sequences (A) used to create 3 homo two stranded templated peptide conjugates (B) derived from Respiratory Syncytial Virus (RSV).

FIG. 7 depicts native sequences (A) used to create 3 hetero two stranded templated peptide conjugates (B) derived from Respiratory Syncytial Virus (RSV).

FIG. 8 depicts native sequences (A) used to create 3 homo two stranded templated peptide conjugates (B) derived from parainfluenza virus 3 (PIV3).

FIG. 9 depicts native sequences (A) used to create 3 hetero two stranded templated peptide conjugates (B) derived from parainfluenza virus 3 (PIV3).

FIG. 10 depicts native sequences (A) used to create 4 hetero two stranded templated peptide conjugates (B) derived from combinations of RSV and PIV3.

FIG. 11 depicts native sequences (A) used to create 3 homo two stranded templated peptide conjugates (B) derived from parainfluenza virus 5 (PIV5).

FIG. 12 depicts native sequences (A) used to create 3 hetero two stranded templated peptide conjugates (B) derived from parainfluenza virus 5 (PIV5).

FIG. 13 depicts native sequences (A) used to create 4 hetero two stranded templated peptide conjugates (B) derived from combinations of RSV and PIV5.

FIG. 14 depicts native sequences (A) used to create 3 homo two stranded templated peptide conjugates (B) derived from parainfluenza virus 4 (PIV5).

FIG. 15 depicts native sequences (A) used to create 3 hetero two stranded templated peptide conjugates (B) derived from parainfluenza virus 4 (PIV5).

FIG. 16 depicts native sequences (A) used to create 4 hetero two stranded templated peptide conjugates (B) derived from combinations of RSV and PIV4.

### DETAILED DESCRIPTION OF THE INVENTION

In one embodiment, the invention comprises a templated conjugate for use in generating an immune response in a subject.

By “subject” is meant a vertebrate, such as a bird or mammal, preferably a human.

A “non-genetically coded” amino acid is an amino acid other than the twenty amino acids used in the genetic code. These twenty genetically coded amino acids are L-alanine, L-arginine, L-asparagine, L-aspartic acid, L-cysteine, L-glutamine, L-glutamic acid, glycine, L-histidine, L-isoleucine, L-leucine, L-lysine, L-methionine, L-phenylalanine, L-proline, L-serine, L-threonine, L-tryptophan, L-tyrosine, and L-valine. Examples of non-genetically coded amino acids useful in the invention are norleucine, alpha-amino-3-guanidino propionic acid, and beta-alanine.

As used herein, a “vaccine” is an immunogenic preparation that is used to induce an immune response in individuals. A vaccine can have more than one constituent that is immunogenic. A vaccine can be used for prophylactic and/or therapeutic purposes. A vaccine does not necessarily have to prevent viral infections. Without being bound by theory, the vaccines of the invention can affect an individual's immune response in a manner such that viral infection occurs in a lesser amount (including not at all) or such that biological or physiological effects of the viral infection are ameliorated when the vaccine is administered as described herein.

As used herein, the term “epitope” refers to a molecule (or association of molecules), containing a region capable of eliciting an immune response and/or containing a region capable of specific binding with an antibody. An epitope may be selected, for example, from a portion of a protein not previously known to bind specifically to an antibody.

“Specific binding” refers to binding with a dissociation constant of no greater than about 10⁻⁶ M, preferably no greater than about 10⁻⁷ M, more preferably no greater than about 10⁻⁸ M, still more preferably no greater than about 10⁻⁹M, yet more preferably no greater than about 10⁻¹⁰ M, or alternatively with affinity of at least about 10⁶/M, preferably at least about 10⁷/M, more preferably at least about 10⁸/M, still more preferably at least about 10⁹/M, yet more preferably at least about 10¹⁰/M.

An “effective amount” or a “sufficient amount” of a substance is that amount sufficient to cause a desired biological effect, such as beneficial results, including clinical results, and, as such, an “effective amount” depends upon the context in which it is being applied. In the context of this invention, an example of an effective amount of a vaccine is an amount sufficient to induce an immune response (e.g., antibody production) in an individual. An effective amount can be administered in one or more administrations.

“Stimulation” or “induction” of an immune response can include both humoral and/or cellular immune responses. In one aspect, it refers to an increase in the response, which can arise from eliciting and/or enhancement of a response as compared to the immune response when no vaccine is given at all.

As used herein, and as well-understood in the art, “treatment” is an approach for obtaining beneficial or desired results, including clinical results. For purposes of this invention, beneficial or desired clinical results include, but are not limited to, alleviation or amelioration of one or more symptoms, diminishment of extent of infection, stabilized (i.e., not worsening) state of infection, amelioration or palliation of the infectious state, and decrease in viral titer (whether detectable or undetectable). “Treatment” can also mean prolonging survival as compared to expected survival if not receiving treatment. Symptoms of viral infection (such as influenza infection) is known to one of skill in the art and can include, but is not limited to, fever, coughing, runny nose, congestion, muscle aches, wheezing, nausea, and fatigue.

“Protective immune response” can include any immune response that provides beneficial or desired clinical results Improving survival rate in an individual can be considered a protective immune response.

In the context of certain vaccine embodiments, “broadly protective” refers to the ability to induce protection against different influenza viruses, e.g., against multiple, serologically distinct influenza virus strains.

A “neutralizing antibody” is understood in the art and for certain examples refers to immunoglobulin from a host animal which is capable of preventing or inhibiting virus infection. For certain embodiments when discussing hemagglutinin glycoprotein structure, the “stem region” is pertinent to the HA2 domain of the influenza HA protein.

As used herein, alkyl groups are monovalent saturated hydrocarbons which can be linear, branched, or cyclic, or a combination thereof. Alkyl groups have the number of carbon atoms specified, e.g., C₁-C₁₂ alkyl groups can have between one and twelve carbon atoms, or, if no number is specified, have about 1 to 8 carbon atoms. Examples of alkyl groups are methyl, ethyl, n-propyl, isopropyl, cyclopropyl, n-butyl, isobutyl, sec-butyl, t-butyl, cyclobutyl, cyclopropyl-methyl, methyl-cyclopropyl, pentyl, cyclopentyl, hexyl, cyclohexyl, heptyl, cycloheptyl, octyl, and cyclooctyl. The alkyl group can be attached to the remainder of the molecule at any position on the alkyl group where a hydrogen can be removed from the corresponding alkane.

As used herein, heteroalkyl groups are monovalent saturated hydrocarbons which can be linear, branched, or cyclic, or a combination thereof, where one or more of the carbon atoms in the group has been replaced by a heteroatom. Heteroatoms include oxygen (—O—), nitrogen (preferably substituted with C₁-C₈ alkyl, for example, —N(CH₃)—), and sulfur (—S—). Heteroalkyl groups have the number of carbon atoms specified, e.g., C₁-C₁₂ heteroalkyl groups can have between one and twelve carbon atoms, or, if no number is specified, have about 1 to about 8 carbon atoms; the number of heteroatoms is not limited, but is preferably from one to three heteroatoms. An example of a heteroalkyl group is —O—CH₂CH₂—O—CH₂CH₂—O—.

As used herein, hydrocarbyl groups are monovalent saturated or unsaturated hydrocarbons which can be linear, branched, or cyclic, or a combination thereof, but excluding aryl and aromatic systems. Hydrocarbyl groups have the number of carbon atoms specified, e.g., C₁-C₁₂ hydrocarbyl groups can have between one and twelve carbon atoms, or, if no number is specified, have about 1 to 8 carbon atoms. Examples of hydrocarbyl groups are methyl, ethyl, ethenyl, acetylenyl, n-propyl, isopropyl, cyclopropyl, n-butyl, isobutyl, sec-butyl, t-butyl, cyclobutyl, 1,3-butadienyl, cyclopropyl-methyl, methyl-cyclopropyl, pentyl, cyclopentyl, hexyl, cyclohexyl, heptyl, cycloheptyl, octyl, and cyclooctyl. The hydrocarbyl group can be attached to the remainder of the molecule at any chemically feasible position on the hydrocarbyl group.

As used herein, the singular form “a”, “an”, and “the” includes plural references unless indicated otherwise. For example, “an” epitope includes one or more epitopes.

### General Methods

The practice of the present invention will employ, unless otherwise indicated, conventional techniques of molecular biology (including recombinant techniques), microbiology, cell biology, biochemistry, nucleic acid chemistry, and immunology, which are within the skill of the art. Such techniques are explained fully in the literature, such as, Molecular Cloning: A Laboratory Manual, second edition (Sambrook et al., 1989) and Molecular Cloning: A Laboratory Manual, third edition (Sambrook and Russel, 2001), (jointly and individually referred to herein as “Sambrook”). Oligonucleotide Synthesis (M. J. Gait, ed., 1984); Animal Cell Culture (R. I. Freshney, ed., 1987); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987, including supplements through 2001); PCR: The Polymerase Chain Reaction, (Mullis et al., eds., 1994); Current Protocols in Immunology (J. E. Coligan et al., eds., 1991); The Immunoassay Handbook (D. Wild, ed., Stockton Press NY, 1994); Bioconjugate Techniques (Greg T. Hermanson, ed., Academic Press, 1996); Methods of Immunological Analysis (R. Masseyeff, W. H. Albert, and N. A. Staines, eds., Weinheim: VCH Verlags gesellschaft mbH, 1993), Antibodies, A Laboratory Manual, (Harlow and Lane, Cold Spring Harbor Publications, New York, 1988); Using Antibodies: A Laboratory Manual (Harlow and Lane, Cold Spring Harbor Laboratory Press, Cold Spring Harbor, N.Y., 1999), Current Protocols in Nucleic Acid Chemistry (Beaucage et al. eds., John Wiley & Sons, Inc., New York, 2000); Protocols for Oligonucleotides and Analogs, Synthesis and Properties (Agrawal, ed., Humana Press Inc., New Jersey, 1993), Vaccines (Plotkin and Orenstein, eds., 4th ed. 2004); and Vaccines (S. Plotkin, 3rd ed. 1999).

### Templated Conjugate Overview

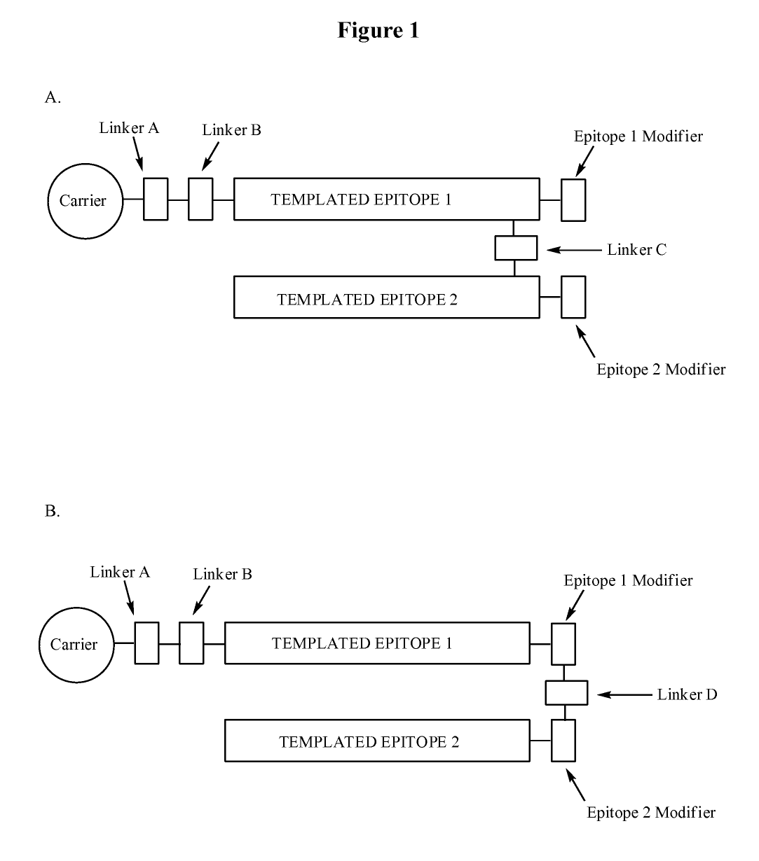

In one embodiment, the invention embraces a templated conjugate such as those shown in FIG. 1. The conjugate comprises a first polypeptide (Templated Epitope 1), a second polypeptide (Templated Epitope 2), an optional Linker A and an optional Linker B, a carrier, an optional Linker C (FIG. 1A), an optional Linker D (FIG. 1B), and an optional Epitope 1 modification and an optional Epitope 2 modification. Each of these elements is discussed in more detail below.

### Carrier

Carriers can be used with the conjugate. Any carrier that is suitable for use in humans or other mammals may be used. In one aspect, the carrier used for the conjugate is typically a protein such as keyhole limpet hemocyanin (KLH), bovine serum albumin (BSA), ovalbumin, tetanus toxoid, cholera subunit B, protein D from H. influenza, or diphtheria toxoid, or a non-proteinaceous moiety such as the polysaccharide alginic acid (alginate). The carrier protein can enhance the immunogenicity of the peptide epitopes. In one aspect, the carrier used is a carrier that is approved by the Food and Drug Administration (FDA) for use in humans.

### Optional Linker A, Optional Linker B

Linker A and Linker B are optional components affixed to the epitope designated Templated Epitope 1. They serve to link Templated Epitope 1, and Templated Epitope 2 associated with Templated Epitope 1, to the carrier protein. They can provide additional functionality; for example, they can act as spacers to ensure that the epitope complex is kept at a sufficient distance from the carrier protein so that the desired coiled coil conformation of the peptide epitopes is not altered by the carrier protein. Inclusion of a non-genetically coded amino acid, such as norleucine or alpha-amino-3-guanidino propionic acid, or another moiety which can be easily assayed without interference from genetically coded amino acids, provides a convenient method of assaying concentration of the conjugate in a given preparation.

In one embodiment, optional Linker A is —CH₂—C(═O)— and optional Linker B is -norleucine-glycine-glycine-(-Nle-Gly-Gly-), where the methylene group of Linker A is covalently attached to the carrier, the carbonyl group of Linker A is covalently attached to the amino group of the norleucine residue of Linker B, and the C-terminal glycine of Linker B is covalently attached to the N-terminal amino group of Templated Epitope 1. If Templated Epitope 1 is prepared by solid phase peptide synthesis, Linker B can be readily incorporated onto Templated Epitope 1 by extending the synthesis to include Nle-Gly-Gly at the N-terminus of Templated Epitope 1.

When Linker A is —CH₂—C(═O)—, it can be readily incorporated by using iodoacetic acid anhydride to attach an iodoacetyl group, I—CH₂—C(═O)—, to Linker B if Linker B is present, or to the N-terminus of Templated Epitope 1 if Linker B is not present. This yields I—CH₂—C(═O)—Nle-Gly-Gly-[Templated Epitope 1], or, if Templated Epitope 2 has been associated with Templated Epitope 1 prior to incorporation of Linker A, this yields I—CH₂—C(═O)—Nle-Gly-Gly-[Templated Epitope 1]-[Templated Epitope 2]. The iodoacetylated complex can then be reacted with a carrier protein containing a nucleophilic moiety, such as a cysteine residue with a free thiol group, resulting in [Carrier protein]-CH₂—C(═O)—Nle-Gly-Gly-[Templated Epitope 1]-[Templated Epitope 2].

Other linkages that can be used include —OOC—(CH₂)ₙ—COO—, where n is an integer from 1 to 12, as Linker A, and —Nle-Gly-Gly- as Linker B. The compound PG_{acid}-OOC—(CH₂)ₙ—COOH, where PG_{acid} is a carboxylic acid protecting group such as t-butyl or benzyl, can be coupled to —Nle-Gly-Gly-[Templated Epitope 1]-[Templated Epitope 2] to form PG_{acid}-OOC—(CH₂)ₙ—COO-Nle-Gly-Gly—[Templated Epitope 1]-[Templated Epitope 2]. The protecting group can then be removed, generating HOOC—(CH₂)ₙ—COO-Nle-Gly-Gly—[Templated Epitope 1]-[Templated Epitope 2], which can be linked to amino groups on the carrier protein using condensing reagents such as 1-ethyl-3-[3-dimethylaminopropyl]carbodiimide (EDC). Exemplary values for n are n=4 (an adipic acid linker) or n=3 (a glutaric acid linker).

Yet another linker that can be used as Linker A is a maleimide-(CH₂)ₙ-carboxylic acid, of the form:

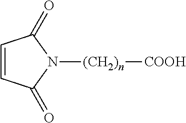

where n is an integer from 1 to 20. These compounds can be readily prepared by reacting a compound of the formula H₂N—(CH₂)ₙ—COOH with maleic anhydride, followed by ring closure (see, for example, U.S. Pat. No. 5,360,914). For a Linker A-Linker B-epitope complex of the form maleimide-(CH₂)ₙ—COO-Nle-Gly-Gly-[Templated Epitope 1]-[Templated Epitope 2], reaction with a carrier protein having free thiol (sulfhydryl) groups will result in attachment of the thiol group(s) to the maleimide moiety.

Another linker that can be used as Linker A is benzoylbenzoic acid, (C₆H₅)—C(═O)—(C₆H₄)—COO—, or

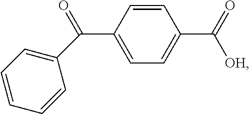

abbreviated as “BB.” This can be readily coupled to -Nle-Gly-Gly-[Templated Epitope 1]-[Templated Epitope 2] to form BB-Nle-Gly-Gly-[Templated Epitope 1]-[Templated Epitope 2]. The benzophenone moiety is activated via UV light to form the triplet diradical —C.(—O.)—, which can then insert into a C—H bond on the carrier molecule.

Preferably, the linkage from the carrier to the epitope complex is “chemically definite.” That is, Linker A (when Linker B is not present), Linker B (when Linker A is not present), Linker A-Linker B (when both are present), or the direct linkage from the carrier to the epitope complex (when Linker A and Linker B are not present) is to a specific functional group or groups on the carrier. In this respect, the iodoacetic acid moiety, the dicarboxylic acid moiety, and the maleimide-carboxylic acid moiety will result in a “chemically definite” reaction with the carrier molecule at a specific function group or groups on the carrier molecule, while the BB moiety can incorporate into a variety of functional groups, and is not “chemically definite.”

### Optional Epitope 1 Modifier, Optional Epitope 2 Modifier

Templated Epitope 1 and Templated Epitope 2 can be optionally modified to incorporate additional desired properties. For example, charged residues such as arginine or lysine, or hydrophilic residues such as histidine, asparagine, or serine, can be added to the C-terminus of the epitopes, which increases the solubility of the complex. In one embodiment, one, two, three, or four arginine residues are added to the C-terminal end of both Templated Epitope 1 and Templated Epitope 2 to enhance the solubility of the complex.

### Coiled-Coil Peptides and Heptad Repeats

The coiled-coil alpha helix motif is often characterized by the heptad repeat:

(abcdefg)

where the letters are used to designate positions in the sequence (that is, “a” is not used to designate alanine, or D-alanine, but rather to designate the first position in the sequence; “b” is not used to designate the aspartic acid/asparagine pair or D-Asx, but rather to designate the second position in the sequence; and so forth). Breaks in heptad repeats—“stutters” (deletion of three amino acids) or “stammers” (deletion of four amino acids)—have been categorized, and other repeat sequences (such as the “hendecad repeat,” equivalent to a heptad repeat followed by a heptad repeat with a stutter), have also been characterized.

The heptad repeat (abcdefg) is often found in the consensus pattern:

(HPPHCPC)

where His a hydrophobic residue, P is a polar residue, and C is a charged residue. The residues in the “a” and “d” positions tend to be hydrophobic; the residues in the “b,” “c,” and “f” positions tend to be polar (hydrophilic), and the residues in the “e” and “g” positions tend to be charged. However, this pattern of polar and charged residues is not absolute, and in the discussion of templated sequences below, it will be seen that the “b,” “c,” “e,” “f,” and “g” need not conform to the HPPHCPC consensus pattern.

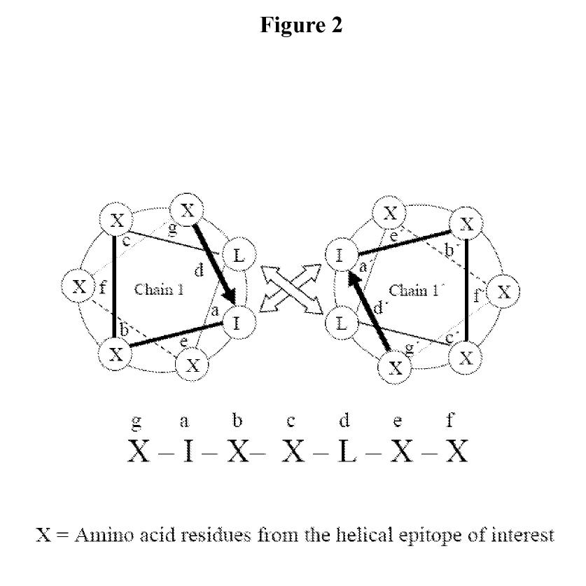

As can be seen from FIG. 2, the heptad repeat of the coiled-coil structure forms an amphiphilic helical structure, with one side of the helix hydrophobic and one side hydrophilic. The helices are arranged such that position “a” and “d” on each helix (these positions are designated a and d on the helix on the left side in FIG. 2, and a′ and d′ in the helix on the right side of FIG. 2), interact with each other, and are relatively shielded from interaction with the solvent. The hydrophobic residues have a thermodynamically favorable interaction with other hydrophobic residues, while the charged and hydrophilic residues have are exposed to solvent. This contributes to stabilization of the coiled-coil structure.

The heptad repeat is a simple sequence motif that determines the oligomerization state of interacting alpha helices. Heptad repeats where isoleucine is in the “a” position and leucine is in the “d” position tend to form dimeric alpha-helical coiled coils. An example of a heptad repeat consensus sequence of 29 amino acids is:

| | |
|---|---|
| IXXLXXXIXXLXXXIXXLXXXIXXLXXXI | (SEQ ID NO: 17) |

a..d...a..d...a..d...a..d...a

In this consensus sequence, there are four complete heptad repeats (28 amino acid residues long), and additionally the first residue (the “a” position) of a fifth heptad repeat, for a total of 29 amino acid residues.

### Heptad Repeat Template Sequences

A heptad repeat sequence, such as the 29-residue heptad repeat sequence described above, can be used as a template for naturally occurring peptide sequences. The naturally occurring peptide sequences can be used to fill in the “X” positions in the template, leaving the isoleucine residues at the “a” positions and the leucine residues at the “d” positions.

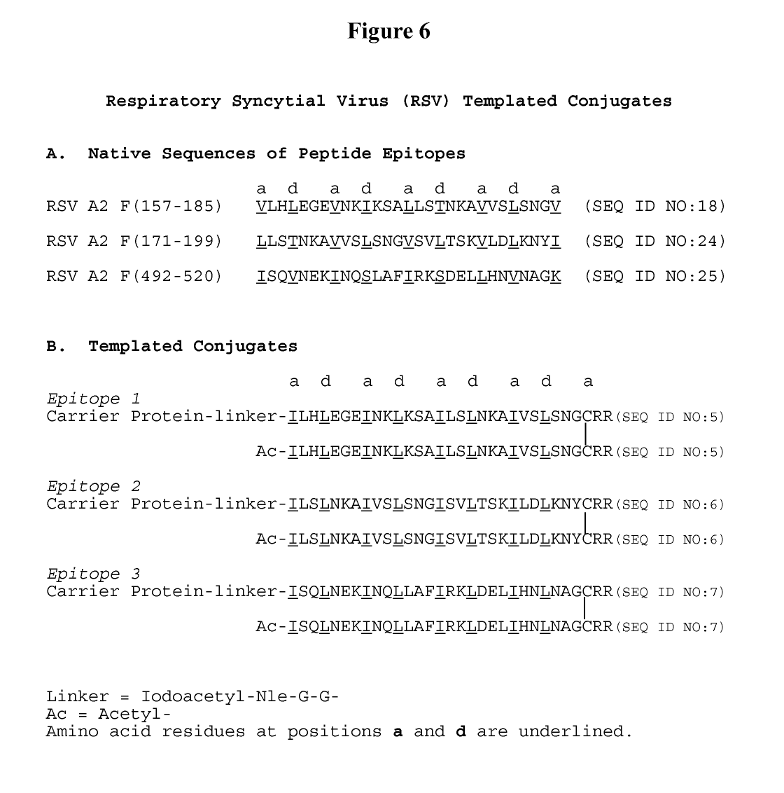

For example, the respiratory syncytial virus sequence RSV A2 F(157-185) (see FIG. 6) VLHLEGEVNKIKSALLSTNKAVVSLSNGV (SEQ ID NO: 18) contains a heptad repeat pattern, as indicated by the underlined residues in the “a” and “d” positions. To “import” this naturally occurring sequence into the template sequence, the underlined residues in the RSV A2 F(157-185) sequence would be replaced with the isoleucine and leucine residues at the “a” and “d” positions, respectively. This process is referred to herein as “templating the naturally occurring sequence”, and the resulting modified sequence is referred to as the “templated sequence,” “templated epitope sequence,” or “Templated Epitope.” This process yields the templated epitope sequence

| | |
|---|---|
| ILHLEGEINKLKSAILSLNKAIVSLSNGI. | (SEQ ID NO: 19) |

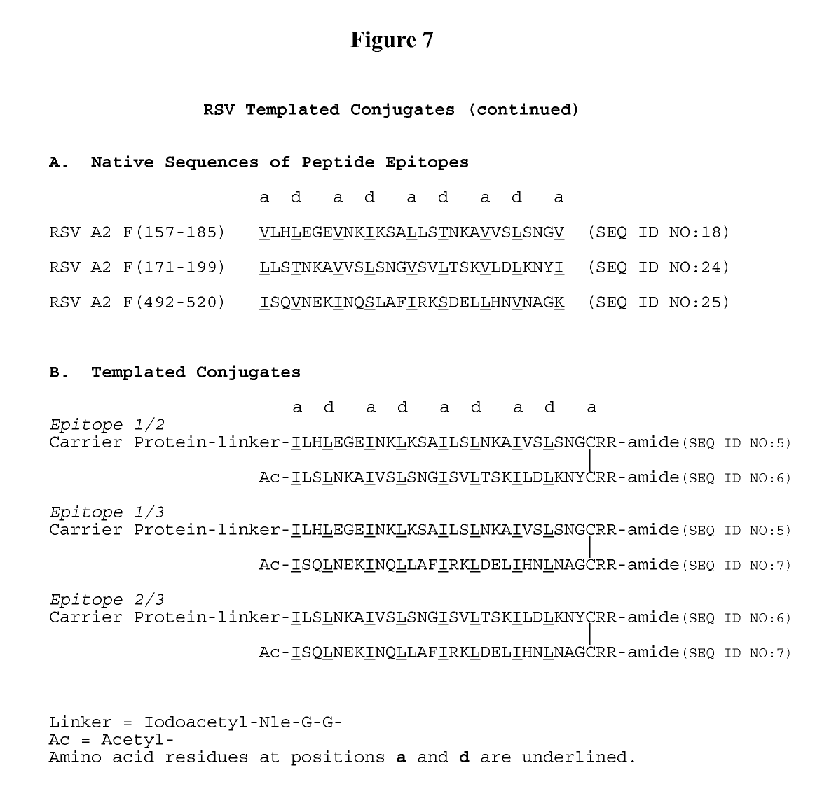

This templated epitope sequence will then favor association with another heptad repeat sequence of approximately equal length, stabilizing both sequences in an alpha-helical coiled-coil configuration. Two identical sequences can be used, as in the templated conjugates in FIG. 6B; or two different sequences can be used, as in the templated conjugates depicted in FIG. 7B. Note that additional modifications have been made to the templated sequences in FIG. 6B and FIG. 7B, such as replacement of the last residue with a cysteine in order to form an inter-chain disulfide bond for greater stability, and the addition of two arginine residues at the C-terminus in order to enhance solubility. One sequence (the “bottom” sequence in the templated conjugates depicted) is acetylated to protect the N-terminal amino group from further modification. The other sequence (the “top” sequence in the templated conjugates depicted) has been extended with the three additional N-terminal amino acids norleucine-glycine-glycine-. The norleucine residue is then reacted with, e.g., iodoacetic acid anhydride to provide an N-terminal iodoacetyl group. The peptide complex is then attached to a carrier protein. These modifications will be discussed in greater detail below.

The residues in the “a” positions of the naturally occurring sequences are replaced with the isoleucine residues of the template sequence, and the residues in the “d” positions of the naturally occurring sequences are replaced with the leucine residues of the template sequence. As can be seen from FIG. 2, positions “a” and “d” of the heptad repeat are on the interior of the coiled-coil structure, while positions “b,” “c,” “e,” “f,” and “g” are on the exterior, solvent-exposed portion of the structure. These exterior positions are much more likely to be recognized by the immune system than the hydrophobic residues buried in the interior of the structure. Using the native sequences for the “b,” “c,” “e,” “f,” and “g” positions therefore provides an epitope in the templated sequence very similar to the epitope present in the naturally occurring protein.

Thus, for templated conjugates incorporating two polypeptides, the first polypeptide comprises the form where n indicates the number of repeating units; n can be an integer between 3 and 20 inclusive, between 3 and 15 inclusive, between 3 and 10 inclusive, or can be 3, 4, 5, 6, 7, 8, 9, 10, 11, 12, 13, 14, 15, 16, 17, 18, 19, or 20. The [I-b₁ᵢ-c₁ᵢ-L-e₁ᵢ-g₁ᵢ] segment repeats n times in the sequence of the first polypeptide. I in each segment is isoleucine, and L in each segment is leucine. In the segments, i is an integer from 1 to n, wherein the value of i is determined by the position of the segment in which it appears, such that the N-terminal segment which appears first in the sequence is assigned a value of i=1, i is incremented by one for each additional segment, and the C-terminal segment is assigned a value of i=n. Thus, a peptide with n=3 would have the sequence [I-b₁₁-c₁₁-L-e₁₁-f₁₁-g₁₁]-[I-b₁₂-c₁₂-L-e₁₂-f₁₂-g₁₂]-[I-b₁₃-c₁₃-L-e₁₃-f₁₃-g₁₃]. Each b, c, e, f, and g in each of the n segments is selected independently of each b, c, e, f, and g amino acid in all other segments of the first polypeptide, and of all segments of the second polypeptide.

Likewise, the second polypeptide comprises the form [I-b₂ᵢ-c₂ᵢ-L-e₂ᵢ-f₂ᵢ-g₂ᵢ]ₙ, where n indicates the number of repeating units; n can be an integer between 3 and 20 inclusive, between 3 and 15 inclusive, between 3 and 10 inclusive, or can be 3, 4, 5, 6, 7, 8, 9, 10, 11, 12, 13, 14, 15, 16, 17, 18, 19, or 20; and the value of n for the second polypeptide is equal to the value of n of the first polypeptide. In the second polypeptide, the segment [I-b₂ᵢ-c₂ᵢ-L-e₂ᵢ-f₂ᵢ-g₂ᵢ] repeats n times. I in each segment is isoleucine, and L in each segment is leucine. As with the segments of the first polypeptide, i is an integer from 1 to n, wherein the value of i is determined by the position of the segment in which it appears, such that the N-terminal segment which appears first in the sequence is assigned a value of i=1, i is incremented by one for each additional segment, and the C-terminal segment is assigned a value of i=n. If n=3 for the first polypeptide, then n=3 for the second polypeptide, and the second polypeptide has the sequence [I-b₂₁-c₂₁-L-e₂₁-f₂₁-g₂₁]-[I-b₂₂-c₂₂-L-e₂₂-f₂₂-g₂₂]-[I-b₂₃-c₂₃-L-e₂₃-f₂₃-g₂₃]. Each b, c, e, f, and g in each of the n segments is selected independently of each b, c, e, f, and g amino acid in all other segments of the second polypeptide, and of all segments of the first polypeptide. It should be noted that the b, c, e, f, and g positions for all segments of one contiguous polypeptide are selected from naturally occurring alpha-helical sequences in pathogens; that is, the sequence of the first templated epitope is derived from a first naturally occurring sequence from a pathogen, and the sequence [I-b₂ᵢ-c₂ᵢ-L-e₂ᵢ-f₂ᵢ-g₂ᵢ]ₙ of the second templated epitope is derived from a second naturally occurring sequence from a pathogen. The first naturally occurring sequence and the second naturally occurring sequence can be the same sequence (to form a homo two-stranded conjugate), or can be different sequences (to form a hetero two-stranded conjugate).

For hetero two-standed conjugates, the b, c, e, f, and g amino acids for use in the segments of the first polypeptide are selected from a first epitope, while the b, c, e, f, and g amino acids for use in the segments of the second polypeptide are selected from an epitope which is different from the epitope of the first polypeptide. In one embodiment, the first and second polypeptides are different from each other. In another embodiment, the first and second polypeptides have less than about 70% sequence homology when the “a” and “d” positions are included in the comparison. In another embodiment, the first and second polypeptides have less than about 90% sequence identity when the “a” and the “d” positions are excluded from the comparison. In another embodiment, the first and second polypeptides have less than about 80% sequence identity when the “a” and the “d” positions are excluded from the comparison. In another embodiment, the first and second polypeptides have less than about 70% sequence identity when the “a” and the “d” positions are excluded from the comparison. In another embodiment, the first and second polypeptides have less than about 60% sequence identity when the “a” and the “d” positions are excluded from the comparison.

### Alignment of Coiled-Coil Peptide Epitopes in Templated Conjugates

In a preferred embodiment, when multiple peptides are used in the conjugates, they are aligned in register. For example, when two peptides are used, their heptad repeats are aligned as follows:

(abcdefg)
(abcdefg)

That is, the “a” residue on one peptide is aligned to interact with the “a” residue on the other strand. When multiple heptad repeats are present, all heptads are aligned in register; for example, for two peptides, where each peptide has four heptad repeats, the peptides would be aligned as follows:

(abcdefgabcdefgabcdefgabcdefg)
(abcdefgabcdefgabcdefgabcdefg)

This alignment stabilizes both helices in the coiled-coil structure. Note that the heptad repeats abcdefg are used to show the alignment of the two peptides, but that the two peptides need not have the identical amino acid sequence. That is, the two peptides depicted may have the same sequence, or may have different sequences, but in both cases, the heptad repeats of one peptide are aligned in register with the heptad repeats of the other peptide.

Either or both peptides can also be stabilized in their alpha-helical form by an intra-chain bridge (see, e.g., Hencheya, L K, Jochima, A L, Aroraa, P S, “Contemporary strategies for the stabilization of peptides in the α-helical conformation,” Current Opinion in Chemical Biology, 2008, 12(6):692-697). Examples of such intra-chain stabilization include one or more lactam bridges between residues (i) and (i+4) in the alpha helix (Houston M E Jr, Gannon C L, Kay C M, Hodges R S, “Lactam bridge stabilization of alpha-helical peptides: ring size, orientation and positional effects,” J. Pept. Sci. 1995, 1(4):274-82), and the “stapled peptide” olefin metathesis method (Schafineister, C E, Po, J, Verdine, G, “An All-Hydrocarbon Cross-Linking System for Enhancing the Helicity and Metabolic Stability of Peptides,” J. Am. Chem. Soc. 2000, 122, 5891-2; Blackwell, H E, Grubbs, R H, “Highly Efficient Synthesis of Covalently Cross-Linked Peptide Helices by Ring-Closing Metathesis,” Angew. Chem. Int. Ed. 1998, 37, 3281-3284).

### Optional Linker C and Optional Linker D: Conformational Stabilization of Two-Stranded Coiled-Coil Structures by Covalent Linkage

The alignment of the two peptides of equal length, or approximately equal length, in the two-stranded coiled-coil stabilizes both helices. Additional stability can be provided by covalently linking the two peptides together via an inter-chain linkage. This can be accomplished via several methods known in the art, for example, by placing cysteine residues at identical locations in the two peptides and forming a disulfide bond between the two peptides; by forming a lactam bridge between a amine-bearing side chain (e.g., a lysine side chain) and a carboxylic acid-bearing side chain (e.g., an aspartic acid or glutamic acid side chain); by olefin metathesis; by linking the carboxy terminals of the peptides together (e.g., using the two amino groups of a diamine compound as the starting points for peptide synthesis), or by other methods. Such a covalent linkage between Templated Epitope 1 and Templated Epitope 2 forms Optional Linker C, as shown in FIG. 1A. In FIG. 1A, Optional Linker C is depicted as located near the C-terminus of Templated Epitope 1 and Templated Epitope 2. However, Optional Linker C can be incorporated anywhere in the sequence of Templated Epitope 1 and Templated Epitope 2, for example, a cysteine residue can be added to the N-terminus of both Templated Epitope 1 and Templated Epitope 2 for formation of a disulfide bridge at the N-terminus. Optional Linker C can also be located between the Epitope 1 Modifier and Epitope 2 Modifier.

An example of formation of a disulfide bridge between peptides is described in Synthetic Example 1.

An example of formation of a linkage at the C-terminus of Templated Epitope 1 and Templated Epitope 2 is described in Synthetic Example 2. 2,3,-diaminopropionic acid is used in the example. It should be appreciated that any diamino compound compatible with solid-phase or solution-phase peptide synthesis can be used, for example, a compound of the form R₁(—NH₂)—R₂-R₃(—NH₂), where R₁ and R₃ can independently be C₁-C₈ hydrocarbyl (preferably C₁-C₈ alkyl), C₁-C₈ heteroalkyl, or HOOC—C₁-C₈ hydrocarbyl (preferably HOOC—C₁-C₈ alkyl), and R₂ can be C₁-C₈ hydrocarbylene (preferably C₁-C₈ alkylene), C₁-C₈ heteroalkylene, or a nonentity. An example of such a compound is HOOC—(CH₂)ₓ—CH(NH₂)—(CH₂)_{y}—CH(NH₂)—(CH₂)_{z}—H, where x, y, and z are independently of each other integers between 0 and 6, inclusive (that is, R₁ is HOOC—CH(NH₂)—, R₂ is C₀-C₆ alkylene, and R₃ is —CH(NH₂)—(C₀-C₆ alkyl)). (For 2,3,-diaminopropionic acid, R₁ is HOOC—CH(NH₂)—, R₂ is a nonentity, and R₃ is —CH₂(NH₂)). Such a compound can be orthogonally protected on the two nitrogen groups (e.g., with a 9-fluorenylmethoxycarbonyl (Fmoc) group on the first nitrogen, and an alloxycarbonyl (Alloc), 4-methyltrityl (Mtt), or 1-(4,4-dimethyl-2,6-dioxocyclohex-1-ylidene)-3-methylbutyl (ivDde) group on the second nitrogen), so that synthesis of one epitope can be carried out on the first nitrogen while the second nitrogen remains protected, and after completion of the synthesis of the first epitope, the second nitrogen can be deprotected and the second epitope synthesized.

When Optional Epitope Modifier Region 1 and Optional Epitope Modifier Region 2 are present, an Optional Linker D can also be placed between them, as shown in FIG. 1B.

In yet another embodiment (not depicted), both Optional Linker C and Optional Linker D can be present.

### Templated Conjugate Details

Returning to the templated conjugate, examples of which are shown in FIG. 1, it can be seen that this template can host a wide variety of epitopes derived from naturally occurring pathogens. For two given antigens 1 and 2, where antigen 1 is to be used as Templated Epitope 1 and antigen 2 is to be used as Templated Epitope 2, “templating” the peptides, or creating a templated conjugate from the two peptides, consists of 1) identifying a heptad repeat region in the first antigen; 2) selecting a region of the first antigen comprising at least one heptad repeat; 3) adapting the selected first antigenic heptad repeat region into the heptad repeat consensus sequence [I-b-c-L-e-f-g]ₙ, where I is isoleucine, L is leucine, and positions “b,” “c,” “e,” “f,” and “g” are derived from the sequence of the selected region of the first antigen, from the respective positions in the heptad repeat of the first antigen, and where n is 3, 4, 5, 6, 7, 8, 9, 10, 11, 12, 13, 14, 15, 16, 17, 18, 19, or 20; 4) using the adapted sequence of the first antigenic heptad repeat region as the sequence for Templated Epitope 1 of the templated conjugate; 5) identifying a heptad repeat region in the second antigen; 6) selecting a region of the second antigen comprising at least one heptad repeat; 7) adapting the selected second antigenic heptad repeat region into the heptad repeat consensus sequence [I-b-c-L-e-f-g]ₙ, where I is isoleucine, L is leucine, and positions “b,” “c,” “e,” “f,” and “g” are derived from the sequence of the selected region of the second antigen, from the respective positions in the heptad repeat of the second antigen, and where n is 3, 4, 5, 6, 7, 8, 9, 10, 11, 12, 13, 14, 15, 16, 17, 18, 19, or 20 and is the same as the value of n in the first heptad repeat consensus sequence adapted from the first antigen; and 8) using the adapted sequence of the second antigenic heptad repeat region as the sequence for Templated Epitope 2 of the templated conjugate; where the heptad repeats of Templated Epitope 1 and Templated Epitope 2 are aligned so that an “a” position of Templated Epitope 1 aligns with an “a” position of Templated Epitope 2, a “b” position of Templated Epitope 1 aligns with a “b” position of Templated Epitope 2, a “c” position of Templated Epitope 1 aligns with a “c” position of Templated Epitope 2, a “d” position of Templated Epitope 1 aligns with a “d” position of Templated Epitope 2, an “e” position of Templated Epitope 1 aligns with an “e” position of Templated Epitope 2, an “f” position of Templated Epitope 1 aligns with an “f” position of Templated Epitope 2, and a “g” position of Templated Epitope 1 aligns with a “g” position of Templated Epitope 2.

After determining the sequences to be used as Templated Epitope 1 and Templated Epitope 2 by the procedure above, the templated conjugate is then synthesized by the following steps, which can be carried out in any order that is chemically feasible: synthesizing Templated Epitope 1 (with the appropriate Epitope 1 Modifier if desired), synthesizing Templated Epitope 2 (with the appropriate Epitope 2 Modifier if desired); covalently linking the peptide epitopes if desired (this step can be carried out at any point in the synthesis as is chemically feasible); adding optional Linker B (if present) and optional Linker A (if present) to Templated Epitope 1 (this step can be carried out before, simultaneously with, or after synthesis of Templated Epitope 1 is completed, as is chemically feasible), and attaching the carrier protein to the epitope-containing fragment of the conjugate to produce the completed templated conjugate.

### Conjugate Design

The hetero two-stranded conjugates, containing two different peptide immunogens, allow the following effective strategies to be used against viruses and other pathogens. The strategies are described in an embodiment for use against enveloped viruses that depend upon Class 1 viral fusion proteins for infection of cells.

Use of a hetero two stranded conjugate to elicit antibodies to two different alpha helical regions in the stem of the same Class 1 viral fusion protein. Targeting two different epitopes on the stem of the fusion protein from a single virus strain will significantly decrease the likelihood of selection of monoclonal antibody-resistant viruses in immunized persons. It will also provide the potential for synergistic protection against the virus by stimulating antibody production from multiple populations of B-cells.

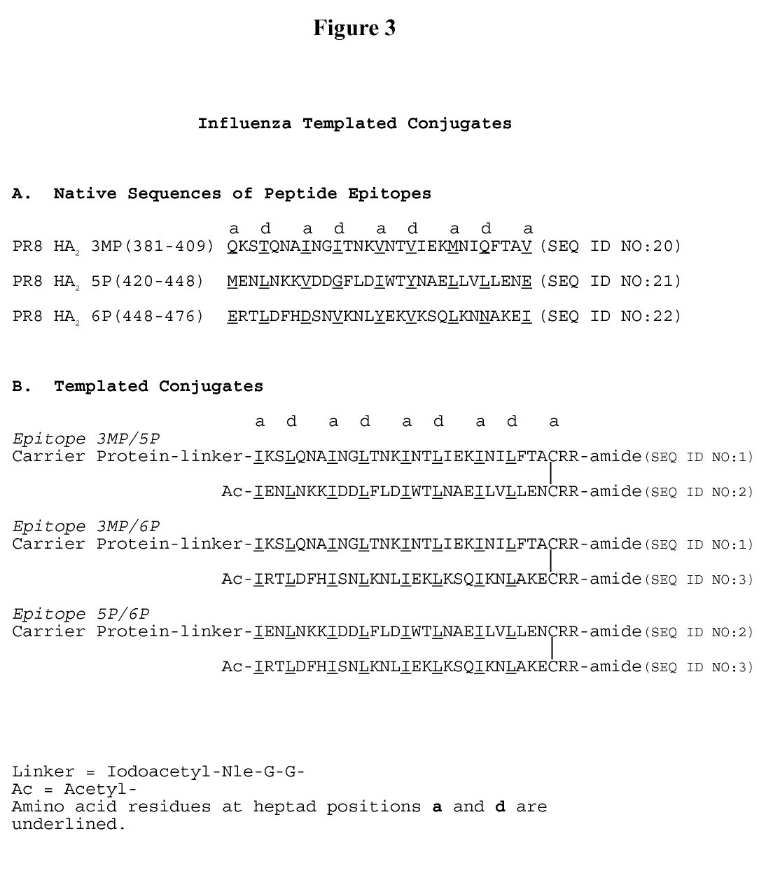

An example of this strategy is a conjugate targeting the stem region of the hemagglutinin (HA) glycoprotein of influenza A virus, pandemic H₁N₁ strain PR8, by synthesizing a two-stranded peptide consisting of one strand of templated peptide 5P with one strand of templated peptide 6P(H1 peptide 5,6); see FIG. 3B, templated conjugate using epitopes 5P/6P. This hetero (HA peptide 5,6) two-stranded conjugate will elicit antibodies to both alpha helical epitopes 5P and 6P in the stem of H1 from strain PR8, allowing the potential for synergistic protection against influenza virus H₁N₁. Additionally, since the selected peptides are highly conserved in H1 proteins of other influenza A virus strains and in related HA proteins such as H2 and H5 in Group 1, this conjugate, when used as a vaccine, has the potential to provide broad cross protection against multiple strains of influenza viruses with different HA types within Group 1.

Use of a hetero two-stranded conjugate to elicit antibodies to the same alpha helical region in the stem of the viral fusion glycoproteins of two different strains of the same virus. Targeting epitopes from the same protein region of different strains of the same virus holds the potential for developing a broadly effective “universal” vaccine protective against many, most, or all strains of a virus. For example, the same epitope in the stem region of the HA glycoproteins of influenza strains H1N1 and H2N1 can be targeted by making a hetero two stranded conjugate consisting of one strand of templated peptide 5P of influenza H1 linked to one strand of templated peptide 5P of influenza H2 (peptide 5P: H1,H2). The 16 known serologically distinct influenza HA proteins form two phylogenetic clusters, Group 1 including H1, H2 and H5 and others, and Group 2 including H3, H7 and others. The selected amino acid sequences in the stem regions of Group 1 HA proteins are significantly different from the corresponding sequences of Group 2 HA proteins. Hetero two stranded conjugates of the same epitope (such as peptide 5P) on influenza H1 and H2 (both from Group 1) have the potential to provide enhanced protection from challenge with both H1 and H2 containing viruses, compared to subjects immunized singly with homo two-stranded conjugates of each of the H1 and H2 viruses. This hetero two-stranded “Peptide 5P: H1,H2” conjugate is expected to provide broader protection against influenza strains with HA proteins in Group 1 than immunization with a homo two-stranded conjugate to an epitope of a single HA type.

Hetero two-stranded templated conjugates targeting the same alpha helical epitope in the stems of more distantly related viruses can also be prepared. For example, a templated conjugate can be prepared from peptide 3 MP of influenza H1 (from Group 1) and from peptide 3 MP of influenza H5 (from Group 2). This will be called “Peptide 3 MP: H1, H5”. This immunogen is expected to elicit antibodies against the selected stem regions of both H1 and H5 proteins, providing protection of subjects against challenge with both H1 and/or H5 strains of influenza virus, and potentially against other influenza A viruses in both Groups 1 and 2. Such a hetero two-stranded immunogen is expected to provide much broader protection against many different influenza strains, with potential effect as the long-sought-after, broadly protective universal influenza vaccine.

Use of a hetero two-stranded conjugate to elicit antibodies to non-homologous alpha helical regions in proteins of two unrelated viruses. This strategy can provide effective immunization against several different common respiratory viruses with a single immunogen. Such a vaccine would be useful against, for example, unrelated respiratory viruses that use class 1 viral fusion proteins for virus infection. There is much merit in making a single vaccine that would effectively target several unrelated respiratory viruses. Respiratory infections are the most common infectious diseases in humans. Many different respiratory viruses can cause similar syndromes, and most of these viruses are very efficiently transmitted in humans. Because hetero two-stranded conjugates where the sequences are derived from alpha helical domains of totally unrelated proteins can be synthesized, immunogens can be made which can simultaneously target key alpha helical regions in the stem domains of two unrelated respiratory viruses. The synthesis of such a hetero two-stranded conjugate is no more difficult than that of the homo two-stranded conjugate.

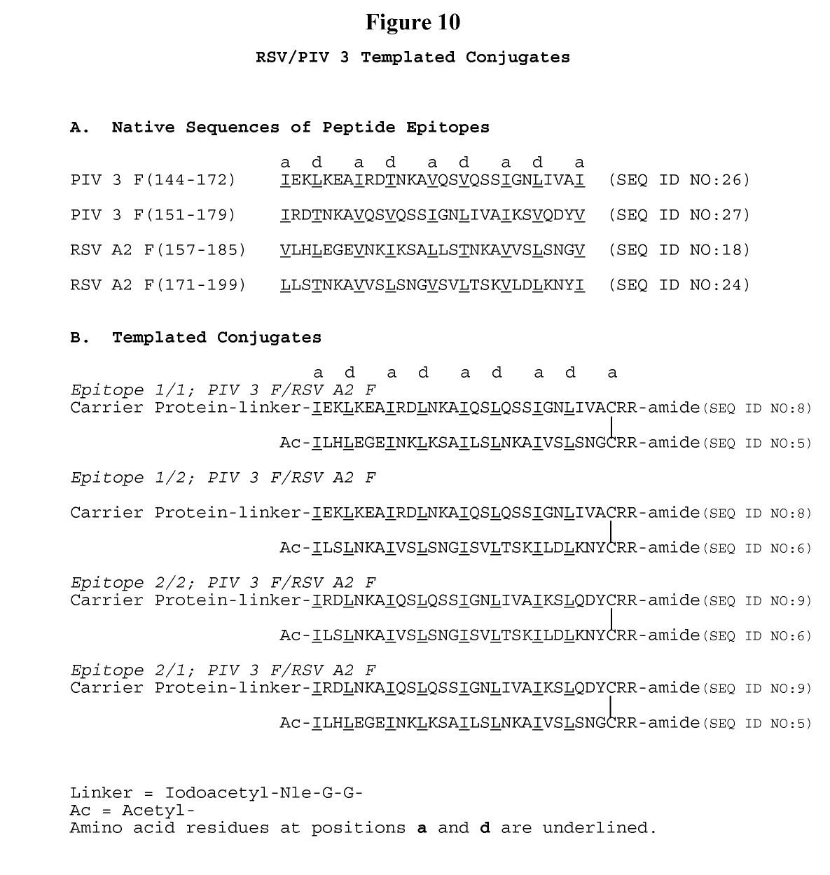

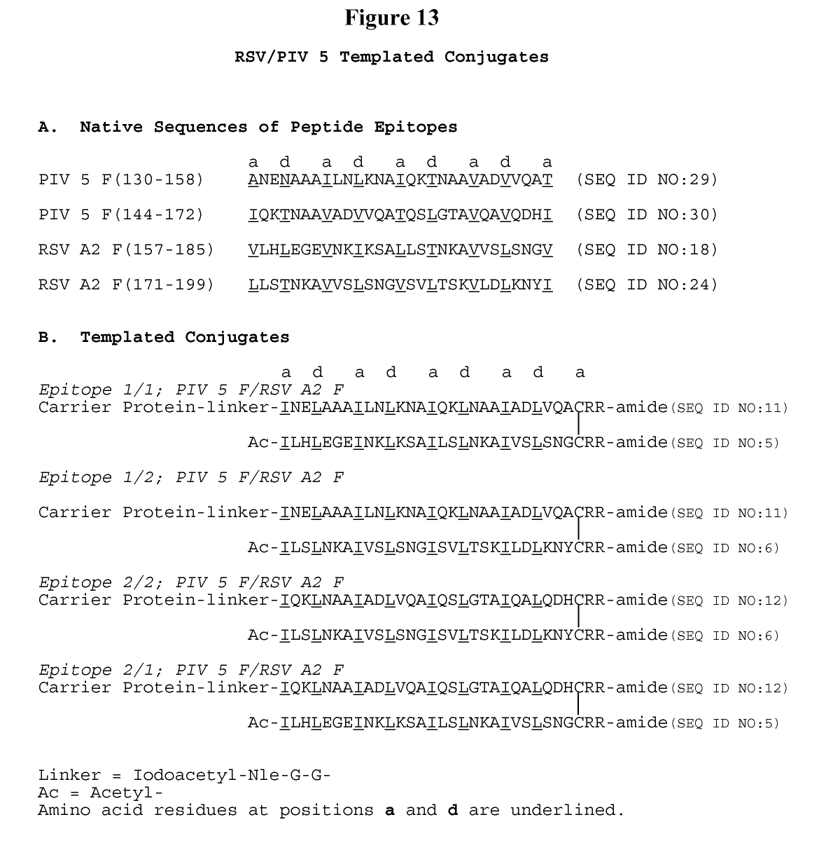

An example of such a vaccine would be a vaccine that targets non-homologous epitopes in stem regions of the F glycoproteins of parainfluenza virus 3 (PIV3) and respiratory syncytial virus (RSV). This vaccine can be constructed by synthesizing a hetero two stranded conjugate consisting of one strand of templated peptide A of PIV3 F protein linked to one strand of templated peptide B of RSV F protein (PIV3 peptide A, RSV peptide B). PIV3 and RSV will be used to show the potential of a templated hetero two-stranded conjugate to provide protection against two unrelated respiratory viruses in subjects. PIV3 and RSV are important respiratory pathogens in infants less than one year of age, and commonly infect and re-infect people of all ages. No active immunization against either of these viruses is currently licensed, and an effective vaccine would be of great value. Many other respiratory pathogens with Class 1 viral fusion proteins can be targeted in this manner, including: influenza B, influenza C, metapneumoviruses, coronaviruses HKU1, 229E, OC43, and NL63, and parainfluenza viruses 1, 2, 4, and 5. Examples of these templated conjugates are shown in FIG. 10B and FIG. 13B.

### Conjugate Configurations

When two peptides (which may be the same templated epitope or different templated epitopes) are present in conjugates of the instant invention, the conjugates can be categorized as follows:

Type I conjugates, comprising one epitope from one virus (and thus homo two-stranded);

Type II conjugates comprising two epitopes from one virus (and thus hetero two-stranded);

Type III conjugates comprising two epitopes from two viruses (and thus hetero two-stranded).

### Influenza Templated Conjugates

Influenza templated conjugates were designed to include two distinct epitopes from influenza A glycoprotein hemagglutinin (HA), from one virus (i.e., a Type II conjugate). The epitopes can be selected from, inter alia, the 29-residue sequences PR8HA₂ 3 MP(381-409) (referred to as 3 MP), PR8HA₂ 5P(420-448) (referred to as 5P), and PR8HA₂ 6P(448-476) (referred to as 6P) (see FIG. 3A). Influenza Virus Templated Epitopes include PR8HA₂ 3 MP(381-409) Templated Epitope 3 MP: IKSLQNAINGLTNKINTLIEKINILFTACRR-amide (SEQ ID NO: 1); PR8HA₂ 5P(420-448) Templated Epitope 5P: IENLNKKIDDLFLDIWTLNAEILVLLENCRR-amide (SEQ ID NO:2); and PR8HA₂ 6P(448-476) Templated Epitope 6P: IRTLDFHISNLKNLIEKLKSQIKNLAKECRR-amide (SEQ ID NO:3). Selecting two out of the set of three provides three different combinations, 3 MP/5P, 3 MP/6P, and 5P/6P, for use in the hetero two-stranded templated conjugates (see FIG. 3B).

### SARS Templated Conjugates

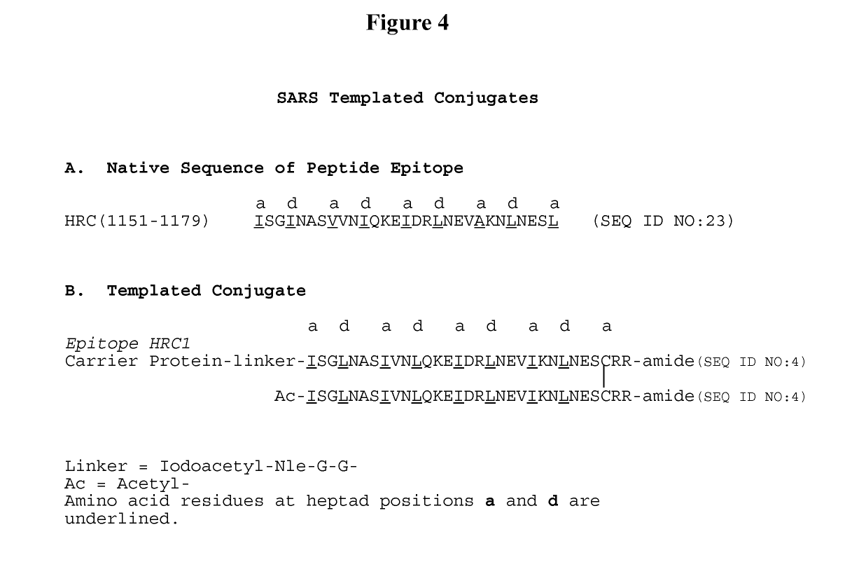

Severe acute respiratory syndrome (SARS) homo two-stranded templated peptide conjugates were designed to include a single epitope from the Spike glycoprotein of the SARS-coronavirus (a Type I conjugate); see FIG. 4A (epitopes) and FIG. 4B (templated conjugates) which uses the SARS HRC(1151-1179) Templated Epitope HRC1: ISGLNASIVNLQKEIDRLNEVIKNLNESCRR-amide (SEQ ID NO:4).

### SARS/Influenza Templated Conjugates

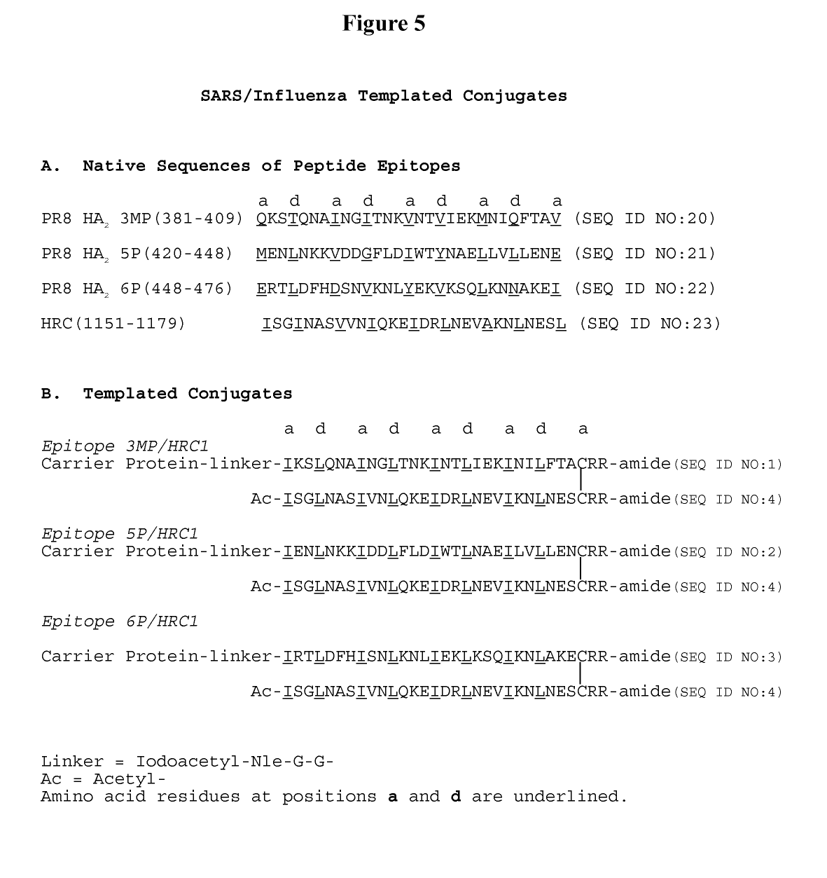

A combined SARS/Influenza templated conjugate was designed, which includes two distinct epitopes from two different viruses, a Type III conjugate. One epitope is derived from influenza A glycoprotein hemagglutinin, and is selected from the 29-residue influenza sequences adapted into Templated Epitope PR8HA₂ 3 MP(381-409), Templated Epitope PR8 HA₂ 5P(420-448), and Templated Epitope PR8HA₂ 6P(448-476), or 3 MP, 5P, and 6P respectively (see above under “Influenza Templated conjugates”). The other epitope, SARS HRC(1151-1179) Templated Epitope HRC1, is derived from the Spike glycoprotein of the SARS-coronavirus. FIG. 5A shows the specific naturally occurring epitopes, while FIG. 5B shows the templated conjugates using the templated epitopes.

### Respiratory Syncytial Virus (RSV) Templated Conjugates

Type I conjugates were designed using a single epitope from Respiratory Syncytial Virus (RSV) F protein. The naturally occurring epitopes selected are RSV A2 F(157-185) (Epitope 1 in FIG. 6A), RSV A2 F(171-199) (Epitope 2 in FIG. 6A), and RSV A2 F(492-520) (Epitope 3 in FIG. 6A). The templated epitope sequences used are RSV A2 F(157-185) Templated Epitope 1: ILHLEGEINKLKSAILSLNKAIVSLSNGCRR-amide (SEQ ID NO:5), RSV A2 F(171-199) Templated Epitope 2: ILSLNKAIVSLSNGISVLTSKILDLKNYCRR-amide (SEQ ID NO:6); RSV A2 F(492-520) Templated Epitope 3: ISQLNEKINQLLAFIRKLDELIHNLNAGCRR-amide (SEQ ID NO: 7) The corresponding Type I templated conjugates are shown in FIG. 6B.

Type II conjugates were also designed using two different epitopes from Respiratory Syncytial Virus (RSV) F protein; the epitopes are shown in FIG. 7A. The combinations possible are Templated Epitopes 1/2, Templated Epitopes 1/3, and Templated Epitopes 2/3; these hetero two-stranded templated peptide conjugates are shown in FIG. 7B.

### Parainfluenza Virus 3 Templated Conjugates

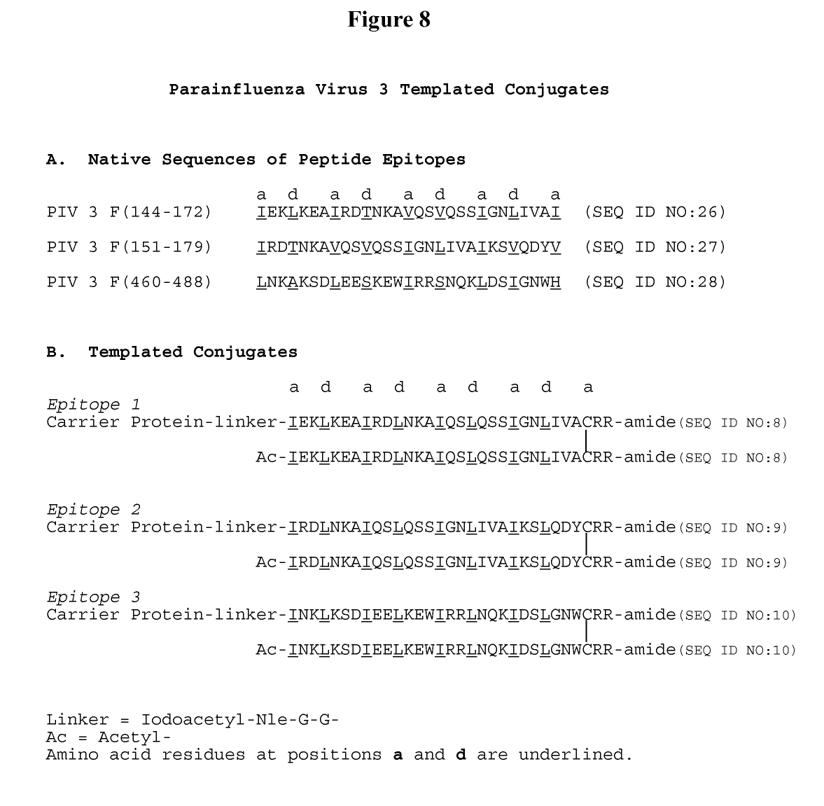

Type I conjugates were designed using a single epitope from the parainfluenza virus (PIV) F protein (AAB48688.1) (see FIG. 8A), using two copies of PIV 3 F(144-172) Templated Epitope 1: IEKLKEAIRDLNKAIQSLQSSIGNLIVACRR-amide (SEQ ID NO: 8); PIV 3 F(151-179) Templated Epitope 2: IRDLNKAIQSLQSSIGNLIVAIKSLQDYCRR-amide (SEQ ID NO:9); or PIV 3 F(460-488) Templated Epitope 3: INKLKSDIEELKEWIRRLNQKIDSLGNWCRR-amide (SEQ ID NO:10), as shown in FIG. 8B.

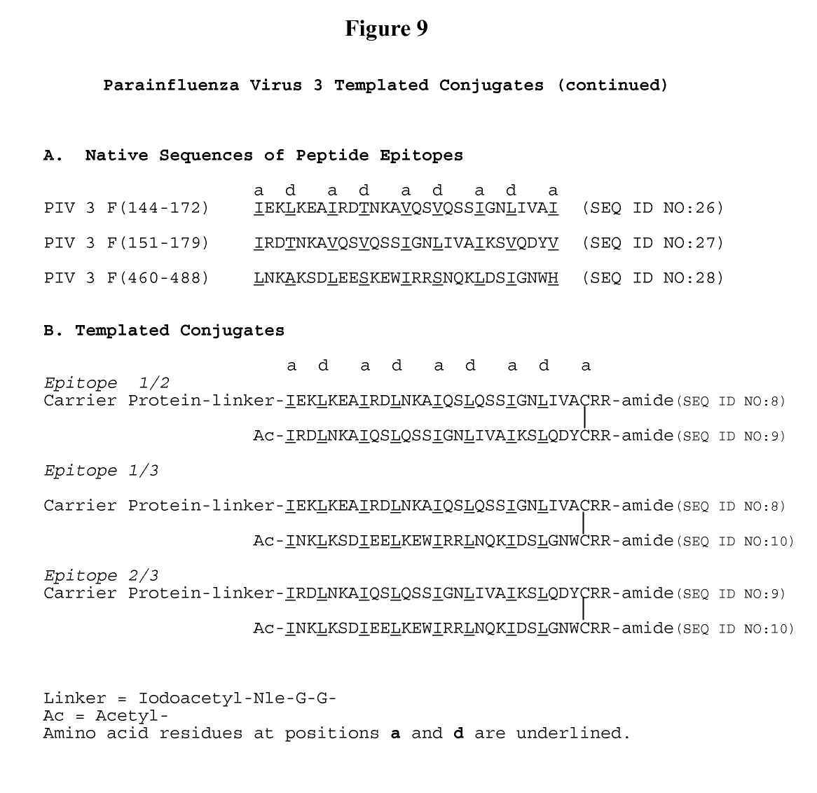

Type II conjugates were designed using two different epitopes from the parainfluenza virus (PIV) F protein (AAB48688.1) (see FIG. 9A), using (Templated Epitope 1)/(Templated Epitope 2), (Templated Epitope 1)/(Templated Epitope 3), or (Templated Epitope 2)/(Templated Epitope 3), as shown in FIG. 9B.

### Respiratory Syncytial Virus (RSV)/Parainfluenza Virus 3 Templated Conjugates

Type III conjugates were designed which combine a templated epitope from RSV with a templated epitope from PIV 3. Naturally occurring epitopes PIV 3 F(144-172), PIV 3 F(151-179), RSV A2 F(157-185), and RSV A2 F(171-199) are shown in FIG. 10A. The templated conjugates [(Templated Epitope PIV 3 F(144-172)/Templated Epitope RSV A2 F(157-185)]; [Templated Epitope PIV 3 F(144-172)/Templated Epitope RSV A2 F(171-199)]; [Templated Epitope PIV 3 F(151-179)/Templated Epitope RSV A2 F(171-199)]; and [Templated Epitope PIV 3 F(151-179)/Templated Epitope RSV A2 F(157-185)] are shown in FIG. 10B.

### Parainfluenza Virus 5 Templated Conjugates

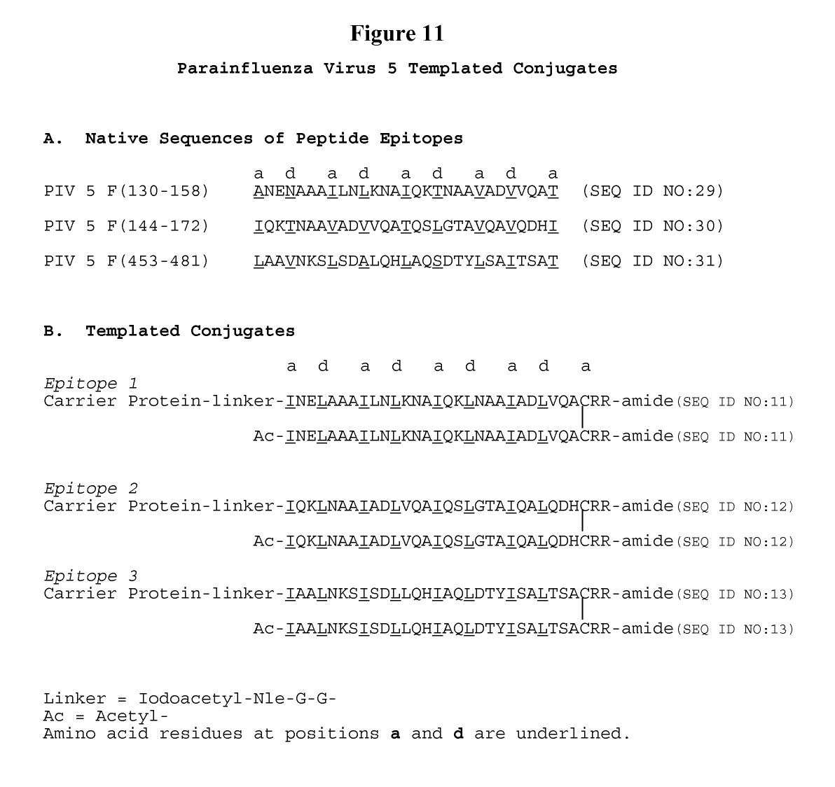

Type I homo two-stranded templated peptide conjugates were designed using single epitopes from the parainfluenza virus (PIV) F protein (YP_{—}138515). See FIG. 11A for the naturally occurring epitopes PIV 5 F(130-158), PIV 5 F(144-172), and PIV 5 F(453-481), and FIG. 11B for the templated conjugates, using PIV 5 F(130-158) Templated Epitope: INELAAAILNLKNAIQKLNAAIADLVQACRR-amide (SEQ ID NO: 11); PIV 5 F(144-172) Templated Epitope: IQKLNAAIADLVQAIQSLGTAIQALQDHCRR-amide (SEQ ID NO:12); and PIV 5 F(453-481) Templated Epitope: IAALNKSISDLLQHIAQLDTYISALTSACRR-amide (SEQ ID NO:13).

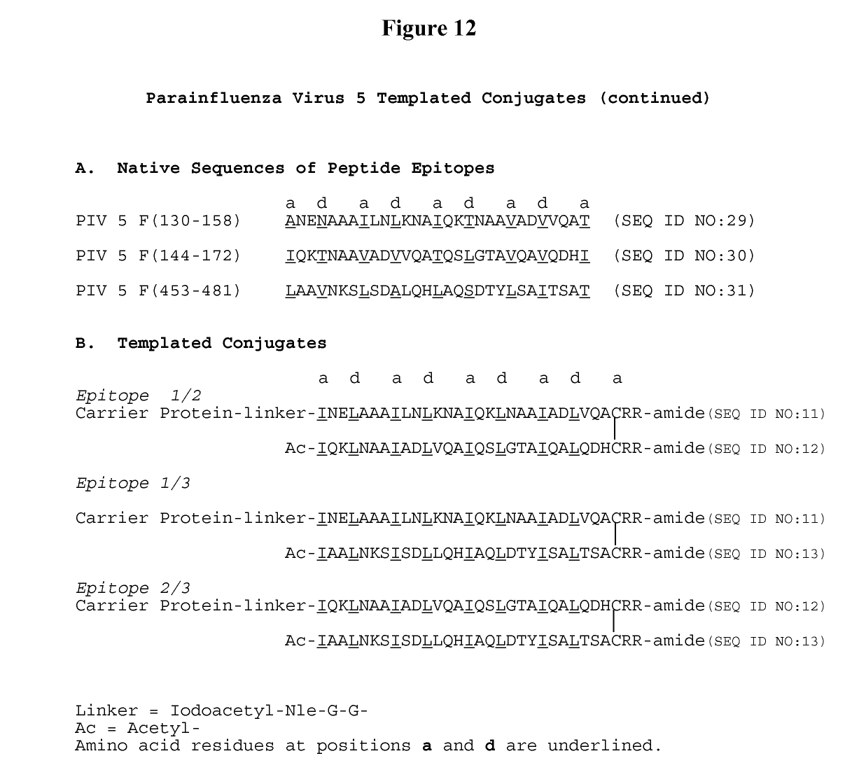

Type II conjugates were designed using two different epitopes from the parainfluenza virus (PIV) F protein. The epitopes are shown in FIG. 12A; the hetero two-stranded templated peptide conjugates are shown in FIG. 12B.

### Respiratory Syncytial Virus (RSV)/Parainfluenza Virus 5 Templated Conjugates

Type III conjugates were designed which combine an epitope from RSV with an epitope from PIV 5. The parainfluenza epitopes are PIV 5 F(130-158) and PIV 5 F(144-172); the RSV epitopes are RSV A2 F(157-185) and RSV A2 F(171-199) (see FIG. 13A). The hetero two-stranded templated peptide conjugates (see FIG. 13B) combine Templated Epitope PIV 5 F(130-158)/Templated Epitope RSV A2 F(157-185); Templated Epitope PIV 5 F(130-158)/Templated Epitope RSV A2 F(171-199); Templated Epitope PIV 5 F(144-172)/Templated Epitope RSV A2 F(171-199); and Templated Epitope PIV 5 F(144-172)/Templated Epitope RSV A2 F(157-185).

### Parainfluenza Virus 4 Templated Conjugates

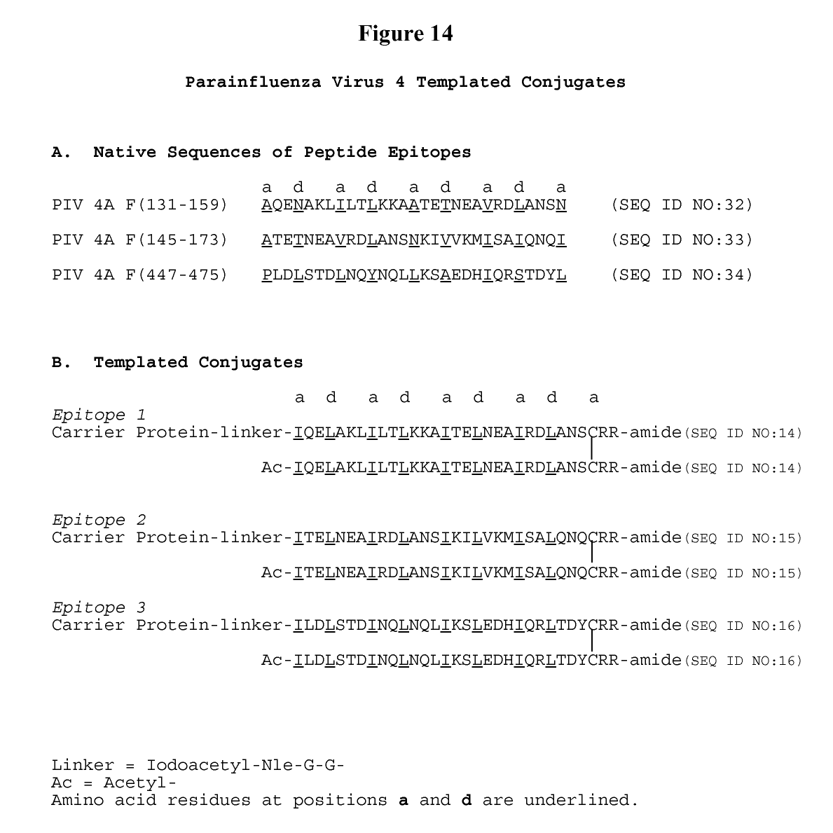

Type I conjugates were designed using single epitopes from the parainfluenza virus (PIV) F protein (BAJ11745). See FIG. 14A for the naturally occurring epitopes PIV 4A F(131-159), PIV 4A F(145-173), and PIV 4A F(447-475). See FIG. 14B for the templated conjugates, using Parainfluenza Virus 4 PIV 4A F(131-159) Templated Epitope: (IQELAKLILTLKKAITELNEAIRDLANSCRR-amide (SEQ ID NO:14)); Parainfluenza Virus 4 PIV 4A F(145-173) Templated Epitope: (ITELNEAIRDLANSIKILVKMISALQNQCRR-amide (SEQ ID NO:15)); and Parainfluenza Virus 4 PIV 4A F(447-475) Templated Epitope: (ILDLSTDINQLNQLIKSLEDHIQRLTDYCRR-amide (SEQ ID NO:16)).

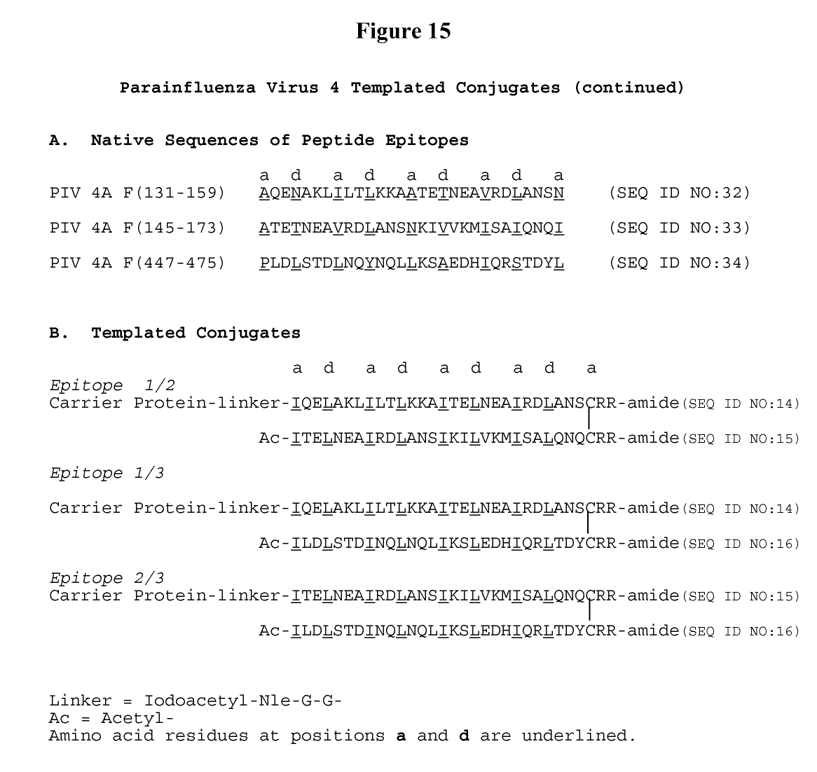

Type II conjugates were designed using two different epitopes from the parainfluenza virus (PIV) F protein. The epitopes are shown in FIG. 15A; the templated conjugates are shown in FIG. 15B.

### Respiratory Syncytial Virus (RSV)/Parainfluenza Virus 4 Templated Conjugates

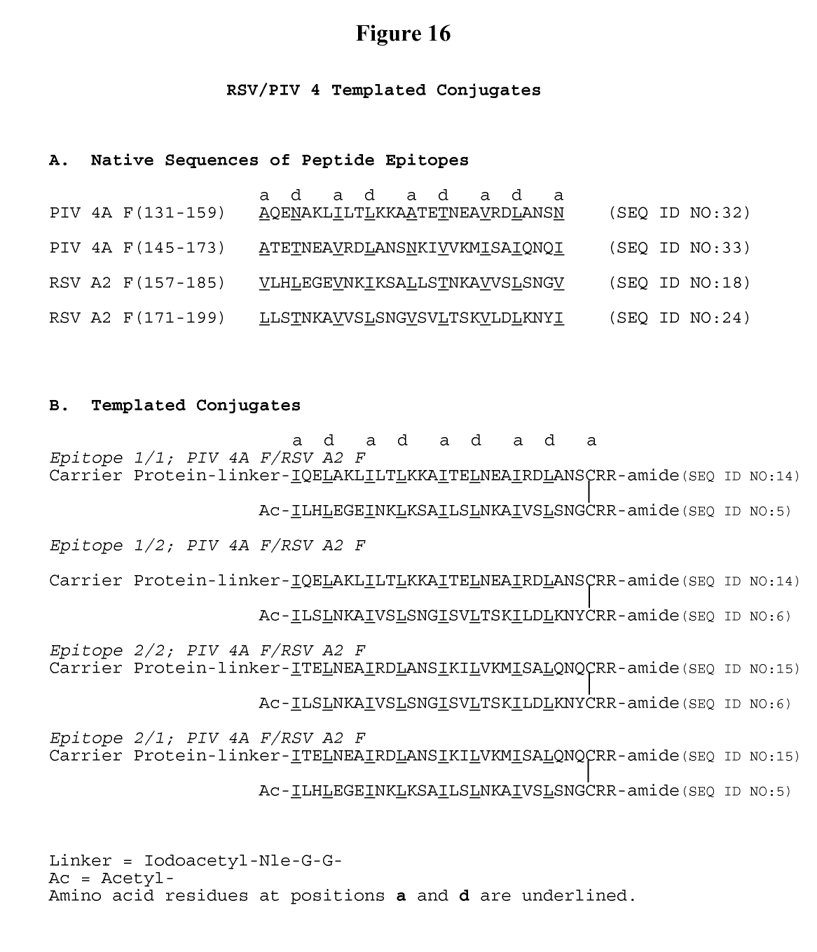

Type III conjugates were designed which combine an epitope from RSV with an epitope from PIV 4. The RSV epitopes are RSV A2 F(157-185) and RSV A2 F(171-199), and the PIV 4 epitopes are PIV 4A F(131-159) and PIV 4A F(145-173) (see FIG. 16A). The templated conjugates (see FIG. 16B) combine Templated Epitope PIV 4A F(131-159)/Templated Epitope RSV A2 F(157-185), Templated Epitope PIV 4A F(131-159)/RSV A2 F(171-199), Templated Epitope PIV 4A F(145-173)/RSV A2 F(171-199), and Templated Epitope PIV 4A F(145-173)/RSV A2 F(157-185).

### Variations of the Sequences

Variations of the templated epitopes can be employed in the conjugates. One of ordinary skill would understand that the description includes variants according to sequence information and sequences which are related by being at a specified level of relative homology or percent identity.

Variants of the templated epitopes can be used which have at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity to the templated epitopes disclosed herein. In an embodiment, the variation is a conservative substitution. In another embodiment, the variant has 1, 2, 3, 4, or 5 changes relative to the templated epitopes disclosed. The substitutions or changes are made in the b, c, e, f, or g locations, while the a and d locations of the heptad repeats are left as found in the sequences of the templated epitopes.

Methods of alignment of sequences for comparison are well known in the art. Thus, the determination of percent identity between any two sequences can be accomplished using a mathematical algorithm. Examples of such mathematical algorithms are the algorithm of Myers and Miller (1988, CABIOS, 4:11); the local homology algorithm of Smith et al. (1981, Adv. Appl. Math., 2:482); the homology alignment algorithm of Needleman and Wunsch (1970, J. Mol. Biol., 48:443); the search-for-similarity-method of Pearson and Lipman (1988, PNAS USA, 85:2444); the algorithm of Karlin and Altschul (1990, PNAS USA, 87:2264), modified as in Karlin and Altschul (1993, PNAS USA, 90:5873). Raghava G P, Barton G J., Quantification of the variation in percentage identity for protein sequence alignments, BMC Bioinformatics. 2006 Sep. 19; 7:415. Raghava G P, Searle S M, Audley P C, Barber J D, Barton G J., OXBench: a benchmark for evaluation of protein multiple sequence alignment accuracy, BMC Bioinformatics. 2003 Oct. 10; 4:47.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988, Gene, 73:237), Higgins et al. (1989, CABIOS, 5:151), Corpet et al. (1988, Nucl. Acids Res., 16:10881), Huang et al. (1992, CABIOS, 8:155), and Pearson et al. (1994, Meth. Mol. Biol., 24:307). The ALIGN program is based on the algorithm of Myers and Miller, supra. The BLAST programs of Altschul et al. (1990, J. Mol. Biol., 215:403; and 1997, Nuc. Acids Res., 25:3389) are based on the algorithm of Karlin and Altschul supra. Software for performing BLAST analyses is publicly available through the National Center for Biotechnology Information (ncbi.nlm.nih.gov on the World Wide Web).

As used herein, “percentage of sequence identity” means the value determined by comparing two optimally aligned sequences over a comparison window, wherein the portion of the polypeptide sequence in the comparison window may include additions or deletions (i.e., gaps) as compared to the reference sequence (which does not include additions or deletions) for optimal alignment of the two sequences. The percentage is calculated by determining the number of positions at which the amino acid residue occurs in both sequences to yield the number of matched positions, dividing the number of matched positions by the total number of positions in the window of comparison, and multiplying the result by 100 to yield the percentage of sequence identity. It should be noted that when two sequences of different length are compared, percent sequence identity is calculated with respect to the length of the shorter sequence.

Naturally occurring amino acid residues are divided into groups based on common side-chain properties: (1) hydrophobic: norleucine, met, ala, val, leu, ile; (2) neutral hydrophilic: cys, ser, thr, asn, gln; (3) acidic: asp, glu; (4) basic: his, lys, arg; (5) residues that influence chain orientation: gly, pro; and (6) aromatic: trp, tyr, phe. Substitution of like amino acids can also be made on the basis of hydrophilicity/hydrophobicity. The hydrophilicity/hydrophobicity scale used in this study is listed as followed: Trp, 33.0; Phe, 30.1; Leu, 24.6; Ile, 22.8; Met, 17.3; Tyr, 16.0; Val, 15.0; Pro, 10.4; Cys, 9.1; His, 4.7; Ala, 4.1; Thr, 4.1; Arg, 4.1; Gln, 1.6; Ser, 1.2; Asn, 1.0; Gly, 0.0; Glu, -0.4; Asp, -0.8 and Lys, -2.0. These hydrophobicity coefficients were determined from reversed-phase chromatography at pH 7 (10 mM PO₄ buffer containing 50 mM NaCl) of a model random coil peptide with a single substitution of all 20 naturally occurring amino acids (see Kovacs, J. M., C. T. Mant and R. S. Hodges. Determination of the intrinsic hydrophilicity/hydrophobicity of amino acid side-chains in peptides in the absence of Nearest-Neighbor or Conformational Effects. Peptide Science (Biopolymers) 84: 283-297 (2006)). We proposed that this HPLC-derived scale reflects the relative difference in hydrophilicity/hydrophobicity of the 20 amino acid side-chains more accurately than previously determined scales (see Mant, C. T., J. M. Kovacs, H. M. Kim, D. D. Pollock and R. S. Hodges. Intrinsic amino acid side-chain hydrophilicity/hydrophobicity coefficients determined by reversed-phase high-performance liquid chromatography of model peptides: comparison with other hydrophilicity/hydrophobicity scales. Peptide Science (Biopolymers) 92: 573-595 (2009)).

Exemplary substitutions for creation of variant polypeptides include those set forth below. While the “b,” “c,” “e,” “f,” and “g” positions are most tolerant of substitutions, a limited number of substitutions can be made at the “a” and “d” positions. Thus, in any of the embodiments of the peptides described herein, one, two, or three of the residues at the “a” or “d” position may be changed from the residues indicated. In one embodiment, one “a” residue is selected from an amino acid other than isoleucine. In one embodiment, two “a” residues are independently selected from amino acids other than isoleucine. In one embodiment, three “a” residues are independently selected from amino acids other than isoleucine. In one embodiment, one “d” residue is selected from an amino acid other than leucine. In one embodiment, two “d” residues are independently selected from amino acids other than leucine. In one embodiment, three “d” residues are independently selected from amino acids other than leucine. In one embodiment, one or two “a” residues are independently selected from an amino acid other than isoleucine and one “d” residue is independently selected from an amino acid other than leucine. In one embodiment, one “a” residue is independently selected from an amino acid other than isoleucine and one or two “d” residues are independently selected from an amino acid other than leucine. The substitutions below are examples of substitutions permitted at the “a,” “b,” “c,” “d,” “e,” “f,” and “g” positions, but the substitutions are not limited to those enumerated in the table below.

| | Substitutions | |
|---|---|---|
| Original residue | Exemplary | Preferred |
| Ala (A) | ser; gly | ser |
| Arg (R) | lys; his | lys |
| Asn (N) | gln; ser; ala | gln |
| Asp (D) | glu; asn | glu |
| Cys (C) | ser; ala | ser; ala |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu, val |
| Leu (L) | norleucine; ile; val; met; ala; phe | ile, norleu |
| Lys (K) | arg | arg |
| Met (M) | leu; ile; norleu | norleu, leu |
| Phe (F) | leu; val; ile; tyr; trp | tyr |
| Pro (P) | ala | ala |
| Ser (S) | thr | thr |
| Thr (T) | ser | ser |
| Trp (W) | tyr; phe | tyr |
| Tyr (Y) | trp; phe; | phe |
| Val (V) | ile; leu; met; phe; norleucine | ile |

### Synthesis of Peptide Epitopes

The peptide epitopes used in the invention can be prepared by chemical or biological methods known in the art. These methods include solid phase peptide synthesis, solution phase peptide synthesis, fragment condensation (either in solution phase or on solid phase), and recombinant DNA technology.

In one embodiment, the peptide epitopes are synthesized by solid phase peptide synthesis (see Stewart and Young, Solid-Phase Peptide Synthesis, 2^{nd} Ed., Pierce Chemical Co. (Rockford, Ill.), 1984; Merrifield, R. B., 1963, J. Am. Chem. Soc. 85:2149-2154; Fmoc Solid Phase Peptide Synthesis: A Practical Approach (Eds. Chan and White), Oxford University Press (New York), 2000). The peptide epitopes can be synthesized and purified separately, and the peptide epitopes can be associated after synthesis and purification of both epitopes have been completed. Alternatively, the peptide epitopes are synthesized either sequentially or simultaneously by synthesis on a linker which aids in maintaining the association of the peptide epitopes. For example, a branched molecule of the form H2N_{β}—(CH2)-CH(N_{α}H₂)—COOH can be attached via its carboxyl group to a solid-phase synthesis resin, such as a crosslinked benzhydrylamine or methylbenzhydrylamine resin. The α and β nitrogens can be orthogonally protected (such as with a Mtt group and an Fmoc group, an ivDde group and an Fmoc group, or with an Alloc group and Fmoc group), and one chain is synthesized to the desired length, followed by synthesis of the other chain to its desired length. The covalently linked two-stranded peptide is then cleaved from the solid phase resin and purified.

The peptides can have routine modifications, such as acetylation of the N-terminal residue, amidation of the C-terminal residue, or both acetylation of the N-terminal residue and amidation of the C-terminal residue.

### Methods of Using Conjugates

Templated conjugates of the invention can be used in various ways. In one aspect, the templated conjugates can be used as a vaccine or immunogenic composition to enhance an individual's immune response (e.g., antibody response). The enhanced immune response is relative to what an individual's immune response would be without exposure to the conjugate. In another aspect of the invention, the conjugates can be used to induce an immune response (e.g., antibody response) in the individual being given the conjugate. For example, an individual's antibody response can be enhanced or induced by generating a greater quantity of antibody and/or antibodies that are more effective at neutralizing virus(es) and/or pathogen(s) of interest. The antibody response can also be enhanced or induced by the generation of antibodies that binds with greater affinity to their targets. In some instances, the antibodies generated are capable to binding to viral strain of various subtypes. Antibodies that are induced or enhanced by the use of the conjugates described herein can be directed to conformational epitopes as well as linear epitopes.

In other aspects, compositions comprising the conjugates as described herein can be used to increase the number of plasma cells and/or memory B cells that can produce antibodies. Methods for measuring specific antibody responses include enzyme-linked immunosorbent assay (ELISA) and are well known in the art. See, e.g., Current Protocols in Immunology (J. E. Coligan et al., eds., 1991). In some aspects, the administration of the conjugates described herein can induce cytokine production (e.g., IL-4, IL-5, and IL-13) that is helpful for antibody production. Cytokine concentrations can be measured, for example, by ELISA. These and other assays to evaluate the immune response to an immunogen are well known in the art. See, for example, SELECTED METHODS IN CELLULAR IMMUNOLOGY (1980) Mishell and Shiigi, eds., W.H. Freeman and Co, and/or Current Protocols in Immunology (J. E. Coligan et al., eds., 1991).

Accordingly, the conjugates described herein can be considered immunogenic compositions. In one aspect, the conjugates can be a component in an immunogenic composition. In another aspect, the conjugates can be a component in a vaccine composition.

In one aspect, the conjugates described herein are used to induce or enhance an individual's immune response (e.g., antibody production or antibody response) such that the viral infection is reduced and in some cases, inhibited. Reduction of viral infection can be at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% from the amount of infection that would have occurred had the immune response not been induced or enhanced. Assays for viral infection are routine and known to one of skill in the art.

In another aspect, the conjugates described herein are used to induce or enhance an individual's immune response (e.g., antibody production or antibody response) such that the viral replication is reduced and in some cases, inhibited. Reduction of viral replication can be at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% from the amount of replication that would have occurred had the immune response not been induced or enhanced. Assays for viral replication are routine and known to one of skill in the art.

### Dosage

The amount of the conjugate, when used as a vaccine, to be administered to an individual in need thereof can be determined by various factors, such as the type of viral infection, the biological and/or physiological response from the individual receiving the vaccine and other factors known to one of skill in the art. As such, the amount of the conjugate to be administered can be adjusted accordingly to achieve the desired beneficial effects. In one aspect, the amount of the conjugate to be used is at least about 1 μg conjugate/kg of the individual. In other aspects, the amount of the conjugate to be used is at least about 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, 21 μg/kg, 22 μg/kg, 23 μg/kg, 24 μg/kg, 25 μg/kg, 26 μg/kg, 27 μg/kg, 28 μg/kg, 29 μg/kg, or 30 μg/kg. In other aspects, the amount of the conjugate to be used is at least about 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg or 100 μg/kg. In other aspects, the amount of the conjugate to be used is about 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, 21 μg/kg, 22 μg/kg, 23 μg/kg, 24 μg/kg, 25 μg/kg, 26 μg/kg, 27 μg/kg, 28 μg/kg, 29 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg or 100 μg conjugate/kg of the individual.

In other aspects, the amount of the conjugate to be used is at most about 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, 21 μg/kg, 22 μg/kg, 23 μg/kg, 24 μg/kg, 25 μg/kg, 26 μg/kg, 27 μg/kg, 28 μg/kg, 29 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg or 100 μg conjugate/kg of the individual. In other aspects, the invention provides for a dosage of range of any of the values given above. For example, the lower limit of the dosage range can be about 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, 21 μg/kg, 22 μg/kg, 23 μg/kg, 24 μg/kg, 25 μg/kg, 26 μg/kg, 27 μg/kg, 28 μg/kg, 29 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg while the upper limit of the dosage range can be 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, 21 μg/kg, 22 μg/kg, 23 μg/kg, 24 μg/kg, 25 μg/kg, 26 μg/kg, 27 μg/kg, 28 μg/kg, 29 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg or 100 μg/kg.

### Modes of Administration

The conjugates described herein can be administered in various ways. In one aspect, the conjugate is administered as an injectable compound. The injection can be by needle injection or needle-free injection (e.g., jet injection). In another aspect, the conjugate is administered as intranasal delivery. The conjugates can also be administered intramuscularly, subcutaneously, intradermally or some combination of all three. These types of injections are known to one of skill in the art.

### Timing of Administration

The conjugates of the invention can be administered with various timing. Timing can be readily determined by one of skill in the art based on the individual's immune parameters. In one aspect, a one-time administration is contemplated. In other aspects, administering the conjugate more than once is contemplated. In these cases, the conjugate can be administered 2, 3, 4, 5, or more times.

If the conjugate is administered more than once, then the interval between the administrations can be of different duration depending on the need of the individual. In some aspects, the interval between the administrations is about 1, 2, 3, 4, 5, 6, or 7 days. In other aspects, the interval between the administrations is about 8, 9, 10, 11, 12, 13, or 14 days. In other aspects, the interval is about 2.5, 3, 3.5, or 4 weeks. In other aspects, monthly intervals are contemplated. The conjugate can be administered upon a determination of need based on the testing of immune parameters in the individuals or based on symptoms experienced by the individual or the individual's exposure to virus(es) and/or other pathogen(s).

### Pharmaceutical Compositions

The conjugates of the invention can be considered as a pharmaceutical composition and or an immunogenic composition. In addition to the other carriers described herein, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like. The conjugate may also be lyophilized using means well known in the art, for subsequent reconstitution and use according to the invention.

Absorption promoters, detergents and chemical irritants (e.g., keritinolytic agents) can be used to enhance the delivery into a target tissue. For reference concerning general principles regarding absorption promoters and detergents which have been used with success in mucosal delivery of organic and peptide-based drugs, see Chien, Novel Drug Delivery Systems, Ch. 4 (Marcel Dekker, 1992).

Examples of suitable nasal absorption promoters in particular are set forth at Chien, supra at Ch. 5, Tables 2 and 3; milder agents are preferred. Suitable agents for use in the method of this invention for mucosal/nasal delivery are also described in Chang, et al., Nasal Drug Delivery, “Treatise on Controlled Drug Delivery”, Ch. 9 and Table 3-4B thereof, (Marcel Dekker, 1992). Suitable agents which are known to enhance absorption of drugs through skin are described in Sloan, Use of Solubility Parameters from-Regular Solution Theory to Describe Partitioning-Driven Processes, Ch. 5, “Prodrugs: Topical and Ocular Drug Delivery” (Marcel Dekker, 1992), and at places elsewhere in the text.

Pharmaceutical compositions can also include vaccines which are formulated for use to induce an immune response to influenza virus. In one aspect, the invention provides a vaccine comprising two templated alpha helical polypeptides of approximately equal length, wherein each polypeptide comprises at least one heptad repeat, and wherein the two polypeptides have less than about 90% sequence identity; a covalent linkage between the two polypeptides; and a carrier protein covalently linked to one of the polypeptides.

The vaccines can also include a carrier as described here. Examples of carriers which may be used include, but are not limited to, alum, microparticles, liposomes, and nanoparticles.

### Adjuvants

The conjugates, immunogens, and vaccines can also be administered with adjuvants. Exemplary adjuvants include alum (Alhydrogel® (Superfos, Denmark; aluminum hydroxide)), and Freund's complete and incomplete adjuvants.

### Sterility

The conjugates, immunogens, and vaccines can be administered as sterile compositions. Sterile pharmaceutical formulations are compounded or manufactured according to pharmaceutical-grade sterilization standards (United States Pharmacopeia Chapters 797, 1072, and 1211; California Business & Professions Code 4127.7; 16 California Code of Regulations 1751, 21 Code of Federal Regulations 211) known to those of skill in the art.

### Kits

The invention further provides kits (or articles of manufacture) comprising a conjugate of the present invention.

In one embodiment, the invention provides a kit comprising both (a) a composition comprising a conjugate as described herein, and (b) instructions for the use of the composition in a subject. In some embodiments, the instructions are on a label. In other embodiments, the instructions are on an insert contained within the kit.

In another embodiment, the invention provides a kit comprising both (a) a composition comprising a conjugate as described herein; and (b) instructions for the administration of the composition to a subject. In some embodiments, the instructions are on a label. In other embodiments, the instructions are on an insert contained within the kit.

In another embodiment, the invention provides a kit comprising both (a) a composition comprising a conjugate as described herein; and (b) instructions for selecting a subject to which the composition is to be administered. In some embodiments, the instructions are on a label. In other embodiments, the instructions are on an insert contained within the kit.

In another embodiment, the invention provides a kit comprising both (a) at least two compositions, each composition comprising a conjugate as described herein; and (b) instructions for selecting one or more compositions to administer to an individual. In some embodiments, the instructions are on a label. In other embodiments, the instructions are on an insert contained within the kit.

### EXAMPLES

### Synthetic Examples

### Synthetic Example 1

### Disulfide Linkage Between Two Peptide Epitopes in a Templated Conjugate

To form disulfide-bridged peptide epitopes, the following procedure is used: 1. Synthesize Epitope 1 (e.g., an acetylated peptide); 2. Cleave and analyze Epitope 1; 3. Purify Epitope 1 by reversed-phase high performance liquid chromatography (RP-HPLC); 4. Analyze fractions, combine and lyophilize; 5. Derivatize Cys of Epitope 1 with DTDP to give Epitope 1 TP; 6. Purify Epitope 1 TP by RP-HPLC; 7. Synthesize Epitope 2 (e.g., can include Nle-G-G linker); 8. Cleave and analyze Epitope 2; 9. Purify Epitope 2 by RP-HPLC; 10. Analyze fractions, combine and lyophilize; 11. Form disulfide bridge Epitope 1 TP and Epitope 2; 12. Purify disulfide bridged Epitope 1-Epitope 2 by RP-HPLC; 13. Analyze fractions, combine and lyophilize; 14. Iodoacetylate the N-terminus of disulfide bridged Epitope 1-Epitope 2; 15. Purify iodoacetylated, disulfide bridged Epitope 1-Epitope 2 by RP-HPLC; 16. Analyze fractions, combine and lyophilize; 17. Conjugate disulfide bridged Epitope 1-Epitope 2 to carrier protein; and 18. Dialyze and lyophilize carrier protein conjugate.

Synthesis of disulfide linker (Optional Linker C) between two cysteine containing peptides. A cysteine-containing peptide is reacted with 2,2′-dithiodipyridine to form the mixed disulfide [Peptide]-S—S-2-pyridine (i.e., [Peptide-S-2-thiopyridine]. The second peptide, containing a free thiol moiety on its cysteine residue, is added to form the disulfide-linked two-stranded peptide (which can be homo-stranded or hetero-stranded).

Step one: the first step of the reaction is carried out with a molar ratio of 1:10 peptide:DTDP. Peptide (e.g., 20 mg) is dissolved in 6 ml reaction solution (3:1 (v/v) acetic acid/H2O). Ten equivalents of 2,2′-dithiopyridine (DTDP) are added in 100 ul DMF and the reaction is stirred at room temperature for four hours. The reaction can be monitored by LC-MS to detect formation of the peptide-TP product. After the reaction is complete, the reaction mixture is diluted in H₂O, followed by purification by HPLC (e.g. reversed-phase HPLC). The collected fraction(s) from the HPLC are freeze dried to give purified peptide-TP.

Step Two: the peptide-TP product from step one and the second peptide containing a free thiol are dissolved in equimolar amounts in 10 ml 40 mM, NH₄Ac, pH 5.5 with 6M GdnHCl. The reaction is incubated at RT for 1 hr. Formation of the two-stranded peptide can be monitored by LC-MS. After the reaction is complete, the two-stranded peptide is purified by HPLC, and the collected fraction(s) are freeze-dried to give the disulfide-linked two-stranded peptide.

Iodoacetylation of disulfide-linked two-stranded peptide. Protecting the reagents, reaction, and products from light, iodoacetic anhydride is dissolved in 1,4-dioxane at a concentration of 100 mM. The disulfide-linked two-stranded peptide is separately dissolved in 100 mM MES, pH 6.0/60% ACN at 0.15 mM. The iodoacetic anhydride solution is slowly added to the peptide solution until reaching the molar ratio 1.2:1, and is incubated at RT for 10 minute. The reaction is monitored by HPLC. After completion, the iodoacetylated is purified by HPLC and lyophilized.

Iodoacetylation can be confirmed by dissolving the iodoacetylated disulfide-linked two-stranded peptide in 6 M GdnHCl, PBS, pH 8.6, and adding DTT at a concentration of 10 mM. DTT will reduce the disulfide bond and also react with the iodoacetyl group. The reaction should yield two peaks when analyzed by LC-MS due to the reduction of the disulfide bond, and the masses should correspond to the separate peptides, where the formerly iodoacetylated peptide has the additional mass of the DTT-acetyl group.

Modification of KLH by Traut's reagent to introduce a free thiol group. KLH is dissolved in 1 ml PBS, pH 8.9; 8 M urea, 5 mM EDTA to prepare a 0.1 mM solution of KLH. Traut's reagent is dissolved in water at 4 mg/ml (28 mM). The Traut's reagent is added to KLH solution at molar ratio 1:40. The mixture is incubated for 1 hr at RT, while protecting from light. Unused Traut's reagent is removed using dialysis.

Conjugation of iodoacetylated covalently linked two-stranded peptide to KLH modified by Traut's reagent. The iodoacetylated covalently linked two-stranded peptide is reacted with the KLH modified by Traut's reagent at a 6:1 two-stranded peptide:KLH ratio in 8 M urea and PBS at RT for up to 48 hours. The progress of the conjugation is followed by reversed-phase HPLC. To terminate the reaction, iodoacetamide in 1 ml water at a concentration of 28 mM is added to the reaction, and the reaction is incubated at RT for 30 mM Dialysis is used to remove free peptide in PBS/8 M urea, 50% ACN/H₂O/0.2% TFA. The sample is freeze-dried to yield the salt-free KLH-peptide conjugate.

Conjugation of iodoacetylated disulfide-linked two-stranded peptide to BSA modified by Traut's reagent. After preparing solution A of BSA, 68 kD (Traut's reagent modified, 0.2 mM, 8 M urea, PBS), and solution B of iodoacetylated two-stranded peptide (0.5 mM, 8 M urea, PBS), the following reactions are conducted:

reaction X: A:B1:5, 20 ul A reacts with 40 ul B in 8 M urea, PBS at RT for 1 hr, 4 hrs, and overnight. (RP-HPLC analysis is used to monitor the conjugation); and reaction R: A:B1:5, 80 ul A reacts with 160 ul B in 8 M urea, PBS at RT for 1 hr, 4 hrs, and overnight. (RP-HPLC analysis is used to monitor the conjugation).
Iodoacetamide in 1 ml water at the concentration of 28 mM is prepared and 100 ul added to the reaction X and R, followed by incubation at RT for 30 mM X and R are combined, dialyzed to remove free peptide in PBS/8 M urea, and then in water/60% ACN/0.2% TFA. Reversed-phase HPLC analysis is used to monitor the removal of free peptide. The sample is freeze-dried to yield salt-free BSA-two-stranded peptide conjugate.

### Synthetic Example 2

### Diaminopropionic Acid Linkage Between Two Peptide Epitopes in a Templated Conjugate

Starting from the following resin-bound diprotected 2,3-diaminopropionic acid reagent:

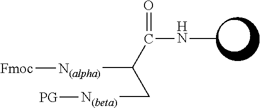

a two-stranded peptide complex covalently linked at the C-terminus can be easily synthesized. The Fmoc group is removed from the alpha-nitrogen of the resin-bound 2,3-diaminopropionic acid and acetylated Epitope 1 is synthesized. After selective deprotection of protecting group PG (PG can be a protecting group such as Alloc, Mtt, or ivDde) from the beta nitrogen of the resin-bound 2,3-diaminopropionic acid, Nle-G-G-Epitope 2 is synthesized. Iodoacetylation of the N-terminus of Nle-G-G-Epitope 2 is performed, followed by cleavage of the peptide from the resin. The peptide complex is purified by reversed-phase HPLC, and the fractions are analyzed, combined, and lyophilized. The peptide complex is then conjugated to a carrier protein, followed by dialysis and lyophilization of the carrier protein-peptide complex conjugate.

### Biological Examples

Generation, purification and characterization of anti-peptide antibodies. For each templated conjugate, three New Zealand white rabbits are immunized at two intramuscular sites. Primary doses contain 50 μg of the conjugate with Freund's complete adjuvant. Boosters at days 7, 28, and 50 contain 50 μg of conjugate, in Freund's incomplete adjuvant. Alternatively, the rabbits are immunized at two intramuscular sites with 50 μg of conjugate with Alhydrogel® aluminum hydroxide adjuvant, with booster immunizations at days 7, 28, and 50. The amount of conjugate used to immunize animals is adjusted based on the response obtained. Sera are collected on day 58, and antibodies are purified with protein G affinity chromatography. The rabbits are euthanized with collection of further samples. Enzyme-linked immunosorbent assays (ELISAs) using plates coated with BSA-peptide conjugates are performed to assess the specificity of the antibodies for their respective coiled-coil templates.

Passive immunization of mice with rabbit IgG directed against templated conjugate and responses to challenge with pathogen. Ten BALB/c mice are passively immunized by the intraperitoneal route with 1 mg per mouse of the antibodies generated in rabbits on days -1, 1 and 3 relative to virus challenge. Control animals receive preimmune rabbit antibody, or buffer alone. On day 0, mice are challenged intranasally with 10 LD₅₀ of pathogen, or buffer. Weight change and mortality are monitored daily for 2 weeks. Virus titers are measured and histopathological studies performed after death or euthanasia of animals.

Biophysical studies of the conjugates. Biophysical studies are conducted to characterize the conjugates. The structures and stability of peptides for use as vaccines is assessed by circular dichroism (CD) spectroscopy in benign buffer (PBS) and in 50% trifluoroethanol (TFE), and also by thermal denaturation profiles. The oligomerization status of templated peptides is examined by analytical ultracentrifugation analysis and size-exclusion chromatography.

Characterization of antibodies. The rabbit antibodies against the immunogens are characterized, for example regarding attributes of peptide-specificity, affinity, and conformation-dependence. Analysis can include the characteristics of whether the antibodies are specific for the immunizing peptides, recognize the alpha-helical conformation of the peptide immunogens, or the native conformation of the entire protein(s) from which the peptide immunogens are derived.

Enzyme-linked immunosorbent assays (ELISA). To characterize the specificity of the rabbit antibodies for the immunizing peptides, ELISA assays are conducted. The conjugate is coated on 96 well polystyrene plates. Five percent BSA is used for blocking. Serial 10-fold dilutions in PBS of rabbit IgG antibodies or IgG from rabbit pre-immune sera are incubated with the bound antigens, and bound IgG is detected with goat anti-rabbit IgG coupled to horseradish peroxidase. Each rabbit anti-peptide IgG or IgG from normal serum is also tested against immunogens and BSA alone to determine the specificity of the antibodies for the synthetic peptide immunogen. A determination of the immunogenicity of each conjugate administered with aluminum hydroxide adjuvant is indicated by the dilution of antibody that gives positive signal in the ELISA.

Similarly, ELISAs are performed to determine whether each antibody recognizes only the conformationally-stabilized, two-stranded, coiled-coiled peptide immunogen or both the immunogen and the single-stranded peptide with native epitope sequence. In this assay, the native epitope sequence is coupled to BSA as a single stranded peptide, which will likely be unstructured since it is removed from the native protein. Some high affinity antibodies specific for an alpha-helical epitope may bind to a single-stranded, unstructured peptide antigen by inducing it to assume a helical conformation. For a particular immunogen, some antibodies generated by the immunogen can recognize both it and the native peptide, but antibodies to other immunogens may be specific for the coiled-coil conformation of the immunogen.

Binding of antibodies to native soluble or anchored trimeric HA protein. The ability of the rabbit antibodies versus pre-immune or naïve rabbit IgG to specifically recognize alpha-helical epitopes in the native protein. This is done by ELISA and/or flow cytometry. The native protein is expressed in appropriate cells and affinity purified. ELISA assays are used to compare binding of the induced rabbit antibodies versus normal rabbit IgG to the target epitope in the native protein.

Assessment of cross-reactivity of anti peptide antibodies for soluble HA trimers. Binding parameters are assessed including with respect to diverse pathogen strains. The binding affinities of antisera to peptide immunogens from different pathogen strains are quantitated using surface plasmon resonance techniques, e.g., with a Biacore biosensor. IgG from immune sera to each of the immunogens or IgG from pre-immune sera is immobilized on the biosensor chip surface. Purified soluble native epitopes from each strain flows over the immobilized antisera. Sensorgrams are generated to indicate on and off rates of binding and the corresponding affinity constant for a given antibody preparation.

Neutralization assays. The antibodies against the peptide immunogens are tested for neutralization of pathogen. A microneutralization assay assesses pathogen neutralization activities of the rabbit anti-peptide antibodies. In an assay, 100 TCID50 of pathogen incubates at 37° C. for 1 hr with equal volumes of 4-fold serial dilutions of antibody (stock IgG concentration, 2 mg/ml). Tissue culture cell lines susceptible to infection with the pathogen of interest are added to each well, and plates are incubated for 18 hours. Virus antigens in alcohol fixed cells are detected by indirect ELISA with a Mab directed against a portion of the virus distinct from the conjugate epitope region. Controls include wells inoculated with medium only, cells with virus only without IgG, and virus mixed with dilutions of IgG from pre-immune rabbit sera. The results demonstrate the ability of antibodies to neutralize pathogen. Combinations of antibody preparations can also be evaluated for neutralization activity. In an embodiment, a combination composition is generated with two or more different antibodies to the peptide-based compounds or conjugate.

Testing is optionally performed for selection of antibody-resistant mutant pathogens. Viruses from the endpoint dilutions of the antibody neutralization experiment are amplified and tested again for neutralization by the same antibody. Viruses with increased resistance to antibody neutralization, if any, can be considered potential antibody escape mutants. The genes from such viruses are studied, e.g., by sequencing, to identify mutations relating to resistance to neutralization with antibodies to certain epitopes. Upon identification of antibody escape mutants, further determinations are made regarding whether these viruses can be neutralized with antibody to a different peptide immunogen. The susceptibility of candidate escape mutant viruses to neutralization with antibody to a different epitope is used as a factor in evaluation of applications for antibody cocktails. Microneutralization assays are also employed for testing induced antibodies against one or more pathogens isolated from humans or animals in geographically distinct areas over several decades. Such isolates show considerable diversity in their neutralization epitopes.

The antibodies induced to a given epitope are evaluated for the ability to block entry of retrovirus pseudotypes containing the Class 1 viral fusion proteins of zoonotic virus strains. Murine retroviruses with proteins of different pathogen strains are made. Using pseudotypes containing different proteins and beta-galactosidase or luciferase reporter genes, antibody-mediated inhibition of transduction of susceptible cells is assessed.

Passive immunization. Antibody preparations arising from the vaccines are tested for efficacy against challenge by pathogen. Passive immunization is demonstrated with rabbit antibody preparations obtained from immunization with the conjugates used as vaccines. Protection is assayed in mice challenged with 10 LD₅₀ units of pathogen.

The protocol for these in vivo protection studies includes intraperitoneal inoculation at days -1, +1 and +3 relative to virus challenge, with IgG from vaccinated rabbits or from pre-immunization controls. Virus-inoculated animals are observed daily with periodic weighing Determinations are made for individual subjects or treatment groups (pre-immune versus immune rabbit IgG for a given immunogen) regarding the mean time to death. Titrations are performed for infectious pathogen in appropriate tissues (e.g., the lungs) at days 2 and 4 after virus inoculation along with titration of rabbit IgG in mouse serum at days 2, 4, 6, 8 and 14 for survivors. Examination of histopathology in relevant tissue (e.g., mouse lungs) is conducted at relevant times post-inoculation.

Active immunization. Mice are actively immunized with the conjugates used as vaccines. The degree of protection or susceptibility to challenge with virulent pathogen is assessed.

Materials and methods. Groups (n=10) of 4-week-old BALB/c mice are immunized intraperitoneally with 100 μl containing 500 μg of aluminum hydroxide gel adjuvant plus either PBS, carrier alone as a control, or 10 μg of conjugate used as vaccine (which may correspond to about 1 μg of peptide). Two or three booster immunizations with the same immunogens are given at 2 week intervals. Blood samples are collected on representative animals from each group just before each boost. Antibody titers to the peptide immunogen are tested by ELISA with the peptide immunogen coupled to BSA. Mouse antibodies are tested in vitro for the ability to neutralize pathogen in microneutralization assays Animals are challenged by inoculation with 10 LD₅₀ units of pathogen. Animals are monitored daily for 14 days after challenge for survival, weight loss, and clinical presentation. Virus titers in appropriate tissue (e.g., lung) are determined on days 2, 4, and 6 after inoculation, and histopathology of appropriate tissue (e.g., lung) is compared in animals immunized with conjugate vs. control animals.

### Hetero Two-Stranded Conjugate Response Comparison to Homo Two-Stranded Conjugate Response

Rabbits and mice are immunized with the hetero two-stranded conjugate composed of Epitope 5P/Epitope 6P of FIG. 3B (HA1 peptide 5P, 6P). The immune response of these animals is compared to the responses of animals immunized singly with homo two stranded conjugates composed of Epitope 5P/Epitope 5P(HA1 peptide 5P, 5P) or Epitope 6P/Epitope 6P(HA1 peptide 6P, 6P), each of which provides partial protection from lethal virus challenge.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A conjugate, comprising:
two templated alpha helical polypeptides of approximately equal length, wherein each polypeptide comprises at least one heptad repeat;
a covalent linkage between the two polypeptides; and
a carrier protein covalently linked to one of the polypeptides;
with the proviso that at least one polypeptide is not derived from an influenza virus protein.

2. The conjugate of claim 1, wherein the two polypeptides have less than about 90% sequence identity.

3. The conjugate of claim 1, wherein the first polypeptide comprises the form:
[I-b₁ᵢ-c₁ᵢ-L-e₁ᵢ-f₁ᵢ-g₁ᵢ]ₙ,
where is a segment that repeats n times in the sequence of the first polypeptide,
where I in each segment is isoleucine,
L in each segment is leucine,
n is an integer of at least 3;
i is an integer from 1 to n, wherein the value of i is determined by the position of the segment in which it appears, such that the N-terminal segment which appears first in the sequence is assigned a value of i=1, i is incremented by one for each additional segment, and the C-terminal segment is assigned a value of i=n;
where each b, c, e, f, and g in each of the n segments is selected independently of each b, c, e, f, and g amino acid in all other segments of the first polypeptide, and of all segments of the second polypeptide;
the b, c, e, f, and g amino acids are selected from an epitope;
the second polypeptide comprises the form:
[I-b₂ᵢ-C₂ᵢ-L-e₂ᵢ-f₂ᵢ-g₂ᵢ]ₙ,
where [I-b₂ᵢ-c₂ᵢ-L-e₂ᵢ-f₂ᵢ-g₂ᵢ] is a segment that repeats n times in the sequence of the second polypeptide,
where I in each segment is isoleucine,
L in each segment is leucine,
n is an integer of at least 3 and is the same as n for the first polypeptide;
i is an integer from 1 to n, wherein the value of i is determined by the position of the segment in which it appears, such that the N-terminal segment which appears first in the sequence is assigned a value of i=1, i is incremented by one for each additional segment, and the C-terminal segment is assigned a value of i=n;
where each b, c, e, f, and g in each of the n segments is selected independently of each b, c, e, f, and g amino acid in all other segments of the second polypeptide, and of all segments of the first polypeptide;
the b, c, e, f, and g amino acids are selected from an epitope which is different from the epitope of the first polypeptide.

4. The conjugate of claim 3, wherein n is an integer between 3 and 15, inclusive.

5. The conjugate of claim 1, wherein each polypeptide comprises at least four heptad repeats.

6. The conjugate of claim 5, wherein a single additional isoleucine residue occurs immediately after the last heptad repeat.

7. The conjugate of claim 1, wherein the conjugate has the form:
[Carrier Moiety]-[Linker A]-[Linker B]-[Templated Epitope 1]-[Epitope 1 Modifier] [Templated Epitope 2]-[Epitope 2 Modifier]
where Linker A, Linker B, Epitope 1 Modifier, and Epitope 2 Modifier are optionally present; and
optionally comprising an additional covalent Linker C between Templated Epitope 1 and Templated Epitope 2;
optionally comprising an additional covalent Linker D between Epitope 1 Modifier and Epitope 2 Modifier, or
optionally comprising an additional covalent Linker C between Templated Epitope 1 and Templated Epitope 2 and an additional covalent Linker D between Epitope 1 Modifier and Epitope 2 Modifier.

8. The conjugate of claim 7, wherein the Epitope 1 Modifier and the Epitope 2 Modifier are present and are selected from hydrophilic, polar, and charged amino acids.

9. The conjugate of claim 7, wherein the [Linker A] moiety is present and is a non-genetically-coded amino acid.

10. The conjugate of claim 7, wherein the [Linker B] moiety is present and is selected from -Gly-, -Gly-Gly-, -(Gly)₃-, and -(Gly)₄-.

11. The conjugate of claim 7, wherein Templated Epitope 1 and Templated Epitope 2 are independently selected from the group consisting of:
Influenza PR8 HA₂ 3MP(381-409) Templated Epitope 3MP
(IKSLQNAINGLTNKINTLIEKINILFTACRR-amide (SEQ ID NO: 1));
Influenza PR8 HA₂ 5P(420-448) Templated Epitope 5P
(IENLNKKIDDLFLDIWTLNAEILVLLENCRR-amide (SEQ ID NO: 2));
Influenza PR8 HA2 6P(448-476) Templated Epitope 6P
(IRTLDFHISNLKNLIEKLKSQIKNLAKECRR-amide (SEQ ID NO: 3));
SARS Coronavirus HRC domain of S2 (1151-1179) Templated Epitope
(ISGLNASIVNLQKEIDRLNEVIKNLNESCRR-amide (SEQ ID NO: 4));
Respiratory Syncytial Virus RSV A2 F(157-185) Templated Epitope
(ILHLEGEINKLKSAILSLNKAIVSLSNGCRR-amide (SEQ ID NO: 5)),
Respiratory Syncytial Virus RSV A2 F(171-199) Templated Epitope
(ILSLNKAIVSLSNGISVLTSKILDLKNYCRR-amide (SEQ ID NO: 6));
Respiratory Syncytial Virus RSV A2 F(492-520) Templated Epitope
(ISQLNEKINQLLAFIRKLDELIHNLNAGCRR-amide (SEQ ID NO: 7));
Parainfluenza Virus 3 PIV 3 F(144-172) Templated Epitope
(IEKLKEAIRDLNKAIQSLQSSIGNLIVACRR-amide (SEQ ID NO: 8));
Parainfluenza Virus 3 PIV 3 F(151-179) Templated Epitope
(IRDLNKAIQSLQSSIGNLIVAIKSLQDYCRR-amide (SEQ ID NO: 9));
Parainfluenza Virus 3 PIV 3 F(460-488) Templated Epitope
(INKLKSDIEELKEWIRRLNQKIDSLGNWCRR-amide (SEQ ID NO: 10));
Parainfluenza Virus 5 PIV 5 F(130-158) Templated Epitope
(INELAAAILNLKNAIQKLNAAIADLVQACRR-amide (SEQ ID NO: 11));
Parainfluenza Virus 5 PIV 5 F(144-172) Templated Epitope
(IQKLNAAIADLVQAIQSLGTAIQALQDHCRR-amide (SEQ ID NO: 12));
Parainfluenza Virus 5 PIV 5 F(453-481) Templated Epitope
(IAALNKSISDLLQHIAQLDTYISALTSACRR-amide (SEQ ID NO: 13));
Parainfluenza Virus 4 PIV 4A F(131-159) Templated Epitope
(IQELAKLILTLKKAITELNEAIRDLANSCRR-amide (SEQ ID NO: 14));
Parainfluenza Virus 4 PIV 4A F(145-173) Templated Epitope
(ITELNEAIRDLANSIKILVKMISALQNQCRR-amide (SEQ ID NO: 15));
and
Parainfluenza Virus 4 PIV 4A F(447-475) Templated Epitope
(ILDLSTDINQLNQLIKSLEDHIQRLTDYCRR-amide (SEQ ID NO: 16))
with the proviso that Templated Epitope 1 and Templated Epitope 2 are not identical.

12. The conjugate of claim 7, wherein Templated Epitope 1 and Templated Epitope 2 are identical, and both of them are selected from one of the sequences from the group consisting of:
SARS Coronavirus HRC domain of S2 (1151-1179) Templated Epitope
(ISGLNASIVNLQKEIDRLNEVIKNLNESCRR-amide (SEQ ID NO: 4));
Respiratory Syncytial Virus RSV A2 F(157-185) Templated Epitope
(ILHLEGEINKLKSAILSLNKAIVSLSNGCRR-amide (SEQ ID NO: 5))
Respiratory Syncytial Virus RSV A2 F(171-199) Templated Epitope
(ILSLNKAIVSLSNGISVLTSKILDLKNYCRR-amide (SEQ ID NO: 6));
Respiratory Syncytial Virus RSV A2 F(492-520) Templated Epitope
(ISQLNEKINQLLAFIRKLDELIHNLNAGCRR-amide (SEQ ID NO: 7));
Parainfluenza Virus 3 PIV 3 F(144-172) Templated Epitope
(IEKLKEAIRDLNKAIQSLQSSIGNLIVACRR-amide (SEQ ID NO: 8));
Parainfluenza Virus 3 PIV 3 F(151-179) Templated Epitope
(IRDLNKAIQSLQSSIGNLIVAIKSLQDYCRR-amide (SEQ ID NO: 9));
Parainfluenza Virus 3 PIV 3 F(460-488) Templated Epitope
(INKLKSDIEELKEWIRRLNQKIDSLGNWCRR-amide (SEQ ID NO: 10));
Parainfluenza Virus 5 PIV 5 F(130-158) Templated Epitope
(INELAAAILNLKNAIQKLNAAIADLVQACRR-amide (SEQ ID NO: 11));
Parainfluenza Virus 5 PIV 5 F(144-172) Templated Epitope
(IQKLNAAIADLVQAIQSLGTAIQALQDHCRR-amide (SEQ ID NO: 12));
Parainfluenza Virus 5 PIV 5 F(453-481) Templated Epitope
(IAALNKSISDLLQHIAQLDTYISALTSACRR-amide (SEQ ID NO: 13));
Parainfluenza Virus 4 PIV 4A F(131-159) Templated Epitope
(IQELAKLILTLKKAITELNEAIRDLANSCRR-amide (SEQ ID NO: 14));
Parainfluenza Virus 4 PIV 4A F(145-173) Templated Epitope
(ITELNEAIRDLANSIKILVKMISALQNQCRR-amide (SEQ ID NO: 15));
or
Parainfluenza Virus 4 PIV 4A F(447-475) Templated Epitope
(ILDLSTDINQLNQLIKSLEDHIQRLTDYCRR-amide (SEQ ID NO: 16)).

13. The conjugate of claim 11, wherein:
Templated Epitope 1 is Influenza PR8HA₂ 3 MP(381-409) Templated Epitope 3 MP and Templated Epitope 2 is SARS Coronavirus HRC domain of S2 (1151-1179) Templated Epitope;
Templated Epitope 1 is Influenza PR8HA₂ 5P(420-448) Templated Epitope 5P and Templated Epitope 2 is SARS Coronavirus HRC domain of S2 (1151-1179) Templated Epitope; or
Templated Epitope 1 is Influenza PR8HA₂ 6P(448-476) Templated Epitope 6P and Templated Epitope 2 is SARS Coronavirus HRC domain of S2 (1151-1179) Templated Epitope.

14. The conjugate of claim 11, wherein:
Templated Epitope 1 is Respiratory Syncytial Virus RSV A2 F(157-185) Templated Epitope and Templated Epitope 2 is Respiratory Syncytial Virus RSV A2 F(171-199) Templated Epitope;
Templated Epitope 1 is Respiratory Syncytial Virus RSV A2 F(157-185) Templated Epitope and Templated Epitope 2 is Respiratory Syncytial Virus RSV A2 F(492-520) Templated Epitope; or
Templated Epitope 1 is Respiratory Syncytial Virus RSV A2 F(171-199) Templated Epitope and Templated Epitope 2 is Respiratory Syncytial Virus RSV A2 F(492-520) Templated Epitope.

15. The conjugate of claim 11, wherein:
Templated Epitope 1 is Parainfluenza Virus 3 PIV 3 F(144-172) Templated Epitope and Templated Epitope 2 is Parainfluenza Virus 3 PIV 3 F(151-179) Templated Epitope;
Templated Epitope 1 is Parainfluenza Virus 3 PIV 3 F(144-172) Templated Epitope and Templated Epitope 2 is Parainfluenza Virus 3 PIV 3 F(460-488) Templated Epitope; or
Templated Epitope 1 is Parainfluenza Virus 3 PIV 3 F(151-179) Templated Epitope and Templated Epitope 2 is Parainfluenza Virus 3 PIV 3 F(460-488) Templated Epitope.

16. The conjugate of claim 11, wherein:
Templated Epitope 1 is Parainfluenza Virus 3 PIV 3 F(144-172) Templated Epitope and Templated Epitope 2 is Respiratory Syncytial Virus RSV A2 F(157-185) Templated Epitope;
Templated Epitope 1 is Parainfluenza Virus 3 PIV 3 F(144-172) Templated Epitope and Templated Epitope 2 is Respiratory Syncytial Virus RSV A2 F(171-199) Templated Epitope;
Templated Epitope 1 is Parainfluenza Virus 3 PIV 3 F(151-179) Templated Epitope and Templated Epitope 2 is Respiratory Syncytial Virus RSV A2 F(171-199) Templated Epitope; or
Templated Epitope 1 is Parainfluenza Virus 3 PIV 3 F(151-179) Templated Epitope and Templated Epitope 2 is Respiratory Syncytial Virus RSV A2 F(157-185) Templated Epitope.

17. The conjugate of claim 11, wherein:
Templated Epitope 1 is Parainfluenza Virus 5 PIV 5 F(130-158) Templated Epitope and Templated Epitope 2 is Parainfluenza Virus 5 PIV 5 F(144-172) Templated Epitope;
Templated Epitope 1 is Parainfluenza Virus 5 PIV 5 F(130-158) Templated Epitope and Templated Epitope 2 is Parainfluenza Virus 5 PIV 5 F(453-481) Templated Epitope; or
Templated Epitope 1 is Parainfluenza Virus 5 PIV 5 F(144-172) Templated Epitope and Templated Epitope 2 is Parainfluenza Virus 5 PIV 5 F(453-481) Templated Epitope.

18. The conjugate of claim 11, wherein:
Templated Epitope 1 is Parainfluenza Virus 5 PIV 5 F(130-158) Templated Epitope and Templated Epitope 2 is Respiratory Syncytial Virus RSV A2 F(157-185) Templated Epitope;
Templated Epitope 1 is Parainfluenza Virus 5 PIV 5 F(130-158) Templated Epitope and Templated Epitope 2 is Respiratory Syncytial Virus RSV A2 F(171-199) Templated Epitope;
Templated Epitope 1 is Parainfluenza Virus 5 PIV 5 F(144-172) Templated Epitope and Templated Epitope 2 is Respiratory Syncytial Virus RSV A2 F(171-199) Templated Epitope; or
Templated Epitope 1 is Parainfluenza Virus 5 PIV 5 F(144-172) Templated Epitope and Templated Epitope 2 is Respiratory Syncytial Virus RSV A2 F(157-185) Templated Epitope.

19. The conjugate of claim 11, wherein:
Templated Epitope 1 is Parainfluenza Virus 4 PIV 4A F(131-159) Templated Epitope and Templated Epitope 2 is Parainfluenza Virus 4 PIV 4A F(145-173) Templated Epitope;
Templated Epitope 1 is Parainfluenza Virus 4 PIV 4A F(131-159) Templated Epitope and Templated Epitope 2 is Parainfluenza Virus 4 PIV 4A F(447-475) Templated Epitope; or
Templated Epitope 1 is Parainfluenza Virus 4 PIV 4A F(145-173) Templated Epitope and Templated Epitope 2 is Parainfluenza Virus 4 PIV 4A F(447-475) Templated Epitope.

20. The conjugate of claim 11, wherein:
Templated Epitope 1 is Parainfluenza Virus 4 PIV 4A F(131-159) Templated Epitope and Templated Epitope 2 is Respiratory Syncytial Virus RSV A2 F(157-185) Templated Epitope;
Templated Epitope 1 is Parainfluenza Virus 4 PIV 4A F(131-159) Templated Epitope and Templated Epitope 2 is Respiratory Syncytial Virus RSV A2 F(171-199) Templated Epitope;
Templated Epitope 1 is Parainfluenza Virus 4 PIV 4A F(145-173) Templated Epitope and Templated Epitope 2 is Respiratory Syncytial Virus RSV A2 F(171-199) Templated Epitope; or
Templated Epitope 1 is Parainfluenza Virus 4 PIV 4A F(145-173) Templated Epitope and Templated Epitope 2 is Respiratory Syncytial Virus RSV A2 F(157-185) Templated Epitope.

21. A method of generating a protective immune response in a subject in need thereof, comprising administering the conjugate of claim 7 to a subject in a sufficient amount to produce the protective immune response.

22. The conjugate of claim 7, wherein the carrier moiety is selected from a protein, keyhole limpet hemocyanin (KLH), bovine serum albumin (BSA), ovalbumin, tetanus toxoid, cholera subunit B, protein D from H. influenza, diphtheria toxoid, a non-proteinaceous moiety, a polysaccharide, or alginic acid (alginate).

23. The conjugate of claim 7, wherein the linkage between the carrier moiety and Linker A (if present), Linker B (if present in the absence of Linker A), or Templated Epitope 1 (if Linker A and Linker B are absent) is chemically definite.

24. A method of inducing an antibody response in an individual in need thereof, the method comprising administering the conjugate of claim 1 to an individual in need thereof in an amount sufficient to induce an antibody response in the individual.

25. The method of claim 24 wherein the antibody response is the production of a neutralizing antibody.

26. A method of inducing an antibody response in an individual in need thereof, the method comprising administering the conjugate of claim 7 to an individual in need thereof in an amount sufficient to induce an antibody response in the individual.

27. The method of claim 26 wherein the antibody response is the production of a neutralizing antibody.
